SEQUENCE LISTING (40 sequences)

SEQ ID NO: 1 - protein, 94 aa, Escherichia coli
SGYTEDEKLNIAKRHLLPKQIERNALKKGELTVDDSAIIGIIRYYTREAGVRGLEREISKLCRKAVKQLLLDKSLKHIEINGDNLHDYLGVQRF

SEQ ID NO: 2 - protein, 97 aa, Saccharomyces cerevisiae
SLLISYESDFKTTLEQAKASLAEAPSQPLSQRNTTLKHVEQQQDELFDLLDQMDVEVNNSIGDASERATYKAKLREWKKTIQSDIKRPLQSLVDSGD

SEQ ID NO: 3 - protein, 106 aa, Drosophila melanogaster
MVYQVKDKADLDGQLTKASGKLVVLDFFATWCGPCKMISPKLVELSTQFADNVVVLKVDVDECEDIAMEYNISSMPTFVFLKNGVKVEEFAGANAKRLEDVIKANI

SEQ ID NO: 4 - protein, 115 aa, Geobacillus kaustophilus
GHMIKGEQKRYSEMTKEELQQEIAMLTEKARKAEQMGMVNEYAVYERKIAMAKAYMLNPADFHPGEIYEIEGAPGEYFKVRYLKGVFAWGWRLKGNGEEEALPISLLRKPNLPQS

SEQ ID NO: 5 - protein, 120 aa, Mus musculus
MIPSITAYSKNGLKIEFTFERSNTNPSVTVITIQASNSTELDMTDFVFQAAVPKTFQLQELSPSSSVVPAFNTGTITQVIKVLNPQKQQLRMRIKLTYNHKGSAMQDLAEVNNFPPQSWQ

SEQ ID NO: 6 - protein, 62 aa, Gallus gallus
MDETGKELVLVLYDYQEKSPRELTVKKGDILTLLNSTNKDWWKIEVNGRQGFVPAAYLKKLD

SEQ ID NO: 7 - protein, 85 aa, Homo sapiens
MGKLPESPKRAEEIWQQSVIGDYLARFKNDRVKALKAMEMTWNNMEKKEKLMWIKKAAEDQKRYERELSEMRAPPAATNSSKKLE

SEQ ID NO: 8 - protein, 93 aa, Artificial Sequence
  Description of Artificial Sequence Synthetic polypeptide
NGYSSEDKLRIAKRHLLPKQIERNALKKGELFVSEFAILGIILFYTDEAGVRGLEREISKLCRKAVKQLLLDKSTTSSHASGFHLHDYLGVMR

SEQ ID NO: 9 - protein, 96 aa, Artificial Sequence
  Description of Artificial Sequence Synthetic polypeptide
SLLDSYKLDFWKTSTNAENSLHEAPSQPLSQRNTTLKHVEQQQDELFDLLDQMDVEVNNSIGRDADRRWYLFELWFWKISIEEEIKRPLQSLVDSG

SEQ ID NO: 10 - protein, 96 aa, Artificial Sequence; identical to SEQ ID NO: 9
  Description of Artificial Sequence Synthetic polypeptide

SEQ ID NO: 11 - protein, 96 aa, Artificial Sequence
  Description of Artificial Sequence Synthetic polypeptide
SLLISYRDDFFTTFWQATQSLKEAPSQPLSQRNTTLKHVEQQQDELFDLLDQMDVEVNNSIGSLASKFAYKFLLDEWKDDIQRDIKRPLQSLVDSG

SEQ ID NO: 12 - protein, 105 aa, Artificial Sequence
  Description of Artificial Sequence Synthetic polypeptide
MVYQAKDFADLFGQAANASGKLIVTVLFATWCGPCKMISPKLVELSTQFADNVVVIKVKALKFEEFARDNNLSSMPVLVFWKNGDEVESFAGANAKRLEDVIKAN

SEQ ID NO: 13 - protein, 103 aa, Artificial Sequence
  Description of Artificial Sequence Synthetic polypeptide
EQKRYSEMTKEELQQEIAMLTEKARKAEQMGMVNEYAVYERKIAMAKAYMLNPADFHPGEIYEIEGAPGEYFKVRYLKGVFAWGWRLKGNGEEEALPISLLRK

SEQ ID NO: 14 - protein, 93 aa, Artificial Sequence; identical to SEQ ID NO: 8
  Description of Artificial Sequence Synthetic polypeptide

SEQ ID NO: 15 - protein, 119 aa, Artificial Sequence
  Description of Artificial Sequence Synthetic polypeptide
MIPSITAYSKNGLKIEFTFERSNTNPSVTVITIQASNSTELDMTDFVFQAAVPKTFQLQELSPSSSVVPAFNTGTITQVIKVLNPQKQQLRMRIKLTYNHKGSAMQDLAEVNNFPPQSW

SEQ ID NO: 16 - protein, 56 aa, Artificial Sequence
  Description of Artificial Sequence Synthetic polypeptide
KELVLVLEEFKSHAPRSLKVKKGDILTILNDDDFSNAKIEVNGRQGFVPLFFAHRI

SEQ ID NO: 17 - protein, 56 aa, Artificial Sequence
  Description of Artificial Sequence Synthetic polypeptide
KLLVLVYDYYSATAPRELTVKKGDILTLLNADLMFYLKIEVNGRQGFVPAFFALWM

SEQ ID NO: 18 - protein, 65 aa, Artificial Sequence
  Description of Artificial Sequence Synthetic polypeptide
LPESPKRAEEIWQQSVIGDYLARFKNDRDRAYASMEFHWRFMSEDLKLMWIKKAAEDQKRYEREL

SEQ ID NO: 19 - protein, 9 aa, Human immunodeficiency virus 1
RKKRRQRRR

SEQ ID NO: 20 - protein, 12 aa, Human immunodeficiency virus 1
YGRKKRRQRRRA

SEQ ID NO: 21 - protein, 13 aa, Human immunodeficiency virus 1
GRKKRRQRRRPPQ

SEQ ID NO: 22 - protein, 16 aa, Drosophila melanogaster
RQIKIWFQNRRMKWKK

SEQ ID NO: 23 - protein, 19 aa, Artificial Sequence
  Description of Artificial Sequence Synthetic peptide
VKRGLKLRHVRPRVTRMDV

SEQ ID NO: 24 - protein, 27 aa, Artificial Sequence
  Description of Artificial Sequence Synthetic peptide
GALFLGFLGAAGSTMGAWSQPKKKRKV

SEQ ID NO: 25 - protein, 21 aa, Artificial Sequence
  Description of Artificial Sequence Synthetic peptide
KETWWETWWTEWSQPKKKRKV

SEQ ID NO: 26 - protein, 18 aa, Mus sp.
LLIILRRRIRKQAHAHSK

SEQ ID NO: 27 - protein, 22 aa, Unknown
  Description of Unknown p14ARF sequence
MVRRFLVTLRIRRACGPPRVRV

SEQ ID NO: 28 - protein, 28 aa, Bos sp.
MVKSKIGSWILVLFVAMWSDVGLCKKRP

SEQ ID NO: 29 - protein, 18 aa, Artificial Sequence
  Description of Artificial Sequence Synthetic peptide
KLALKLALKALKAALKLA

SEQ ID NO: 30 - protein, 27 aa, Artificial Sequence
  Description of Artificial Sequence Synthetic peptide
GWTLNSAGYLLGKINLKALAALAKKIL

SEQ ID NO: 31 - protein, 28 aa, Unknown
  Description of Unknown Azurin sequence
LSTAADMQGVVTDGMASGLDKDYLKPDD

SEQ ID NO: 32 - protein, 26 aa, Artificial Sequence
  Description of Artificial Sequence Synthetic peptide
DPKGDPKGVTVTVTVTVTGKGDPKPD

SEQ ID NO: 33 - protein, 24 aa, Artificial Sequence
  Description of Artificial Sequence Synthetic peptide
RRIRPRPPRLPRPRPRPLPFPRPG

SEQ ID NO: 34 - protein, 17 aa, Artificial Sequence
  Description of Artificial Sequence Synthetic peptide
CSIPPEVKFNKPFVYLI

SEQ ID NO: 35 - protein, 6 aa, Artificial Sequence
  Description of Artificial Sequence Synthetic peptide
PFVYLI

SEQ ID NO: 36 - protein, 15 aa, Artificial Sequence
  Description of Artificial Sequence Synthetic peptide
SDLWEMMMVSLACQY

SEQ ID NO: 37 - protein, 93 aa, Artificial Sequence
  Description of Artificial Sequence Synthetic polypeptide
NGYWSEDKERIAKRHLLPKQIERNALKKGELKVWEFAIFGIILFYTWEAGVRGLEREISKLCRKAVKQLLLDKSTTRSWASGFHLHDYLGVMR

SEQ ID NO: 38 - protein, 65 aa, Artificial Sequence
  Description of Artificial Sequence Synthetic polypeptide
LPESPRFAQKIWQQSVIGDYLARFKNDRVKALKAMEDSSNNSSMHRFAEFFAKAFEDLHRYLEEL

SEQ ID NO: 39 - protein, 274 aa, Artificial Sequence
  Description of Artificial Sequence Synthetic polypeptide
MSVSAGEKPPRLYDRRSIFDAVAQSNCQELESLLPFLQRSKKRLTDSEFKDPETGKTCLLKAMLNLHNGQNDTIALLLDVARKTDSLKQFVNASYTDSYYKGQTALHIAIERRNMTLVTLLVENGADVQAAANGDFFKKTKGRPGFYFGELPLSLAACTNQLAIVKFLLQNSWQPADISARDSVGNTVLHALVEVADNTVDNTKFVTSMYNEILILGAKLHPTLKLEEITNRKGLTPLALAASSGKIGVLAYILQREIHEPECRHAAAHHHHHH

SEQ ID NO: 40 - protein, 248 aa, Rattus norvegicus
LYDRRSIFDAVAQSNCQELESLLPFLQRSKKRLTDSEFKDPETGKTCLLKAMLNLHNGQNDTIALLLDVARKTDSLKQFVNASYTDSYYKGQTALHIAIERRNMTLVTLLVENGADVQAAANGDFFKKTKGRPGFYFGELPLSLAACTNQLAIVKFLLQNSWQPADISARDSVGNTVLHALVEVADNTVDNTKFVTSMYNEILILGAKLHPTLKLEEITNRKGLTPLALAASSGKIGVLAYILQREIH

(19) United States
(12) Patent Application Publication
(10) Pub. No.: US 2024/0166695 A1
(43) Pub. Date: May 23, 2024

(54) PEPTIDIC POSITIVE ALLOSTERIC MODULATORS TARGETING TRPV1

(71) Applicant: The Regents of the University of California, Oakland, CA (US)

(72) Inventors: Jie Zheng, Oakland, CA (US); Fan Yang, Oakland, CA (US)

(73) Assignee: The Regents of the University of California, Oakland, CA (US)

(21) Appl. No.: 18/283,572

(22) PCT Filed: Mar. 25, 2022
(86) PCT No.: PCT/US2022/021961

§ 371 Date: Sep. 22, 2023

Related U.S. Application Data
(60) Provisional application No. 63/166,533, filed on Mar. 26, 2021.

(51) Int. Cl. C07K 14/005; A61K 38/16; A61K 45/06; A61P 25/04; C07K 14/00; C12N 7/00

(52) U.S. Cl. CPC C07K 14/005; A61K 38/162; A61K 45/06; A61P 25/04; C07K 14/001; C12N 7/00; C07K 2319/75; C12N 2740/16022; C12N 2740/16052; C12N 2740/16071

(57) ABSTRACT

Peptidic positive allosteric modulators of TRPV1 are provided. Methods for the design of TPRV1 allosteric modulators are also described, as well as methods for the treatment of conditions such as pain, pruritus, and cancer.

### CROSS-REFERENCES TO RELATED APPLICATIONS

The present application claims priority to U.S. Provisional Pat. Appl. No. 63/166,533, filed Mar. 26, 2021, which application is incorporated herein by reference in its entirety.

### STATEMENT AS TO RIGHTS TO INVENTIONS MADE UNDER FEDERALLY SPONSORED RESEARCH AND DEVELOPMENT

This invention was made with Government support under Grant Nos. R01NS072377 and R01NS103954 awarded by the National Institutes of Health. The Government has certain rights in the invention.

### BACKGROUND OF THE INVENTION

Chronic pain is more than an unpleasant feeling, it can be so devastating that not only the quality of life in patients is drastically lowered, but also enormous social-economical costs are imposed. For instance, in the United States alone, over 100 million adults suffer from chronic pain with an annual economic cost of nearly 600 billion dollars (1). Though analgesics such as opioids and non-steroidal anti-inflammatory drugs are available, their low efficacy against chronic pain, side effects and the complex nature of pain demand developments of novel analgesic drugs.

The TRPV1 ion channel is a prototypical sensor involved in nociception (2), making it a promising target for pain managements (3). Indeed, genetically knocking out this channel leads to much reduction in thermal hyperalgesia (4). Antagonizing TRPV1 pharmacologically also effectively alleviates dental, rectal and thermal pain (5, 6). However, because TRPV1 channel is a polymodal receptor activated by heat and involved in body temperature regulation, systematic blockade of this channel incurred substantial hyperthermia in clinical trials, thus impeding further drug developments (5, 7). TRPV1 agonists like resiniferatoxin (RETX) potently ablate TRPV1-expressing neurons in the dorsal root and trigeminal ganglia by inducing calcium overload in these neurons (8), but such analgesia is irreversible so that its application is limited to intractable cancer pain (9).

### BRIEF SUMMARY OF THE INVENTION

Provided herein are peptidic positive allosteric modulators of TRPV1. The allosteric modulators include a peptide containing two hotspot amino acid residues (e.g., aromatic amino acid residues such as phenylalanine, tyrosine, and tryptophan), wherein the alpha carbon atoms in the two hotspot amino acid residues are within 5-10 Å of each other upon folding of the peptide under physiological conditions.

In some embodiments, the allosteric modulators may be prepared by a processing comprising:
i) assessing shape complementarity, binding energy, and solvent accessible surface area of a candidate peptide upon docking to a surface of a TRPV1 ankyrin repeat domain (ARD) in silico;
ii) assessing ab initio refolding of the candidate peptide;
iii) evolving the candidate peptide via in silico affinity maturation to define a mature allosteric modulator;
iv) expressing or synthesizing the mature allosteric modulator, thereby preparing the TRPV1 allosteric modulator.

Methods for treating conditions associated with TRPV1, such as pain, pruritus, and cancer, are also provided. The methods include administering an effective amount of a TRPV1 allosteric modulator as described herein, or a nucleic acid encoding the allosteric modulator to a subject in need thereof.

### BRIEF DESCRIPTION OF THE DRAWINGS

FIG. 1 shows a flow chart summarizing the design of peptidic TRPV1 positive allosteric modulators.

FIG. 2A shows the computational design of protein binders to the ARD of TRPV1. Sideview of the structure of TRPV1 channel shown as cylinders (PDB ID: 3J5P). The transmembrane domains are shaded in green. The intracellular ARD is highlighted in orange.

FIG. 2B shows two histidine residues docked to the concave surface of ARD serve as the hot spots to anchor the binder to the ARD.

FIG. 2(C shows a multi-metric evaluation of initial binder designs with shape complementarity, SASA and ddg.

FIG. 2D shows refolding of the candidate designs with ab initio modeling. The designed structures serve as the reference to calculate and plot the root mean±square deviation (RMSD) of models versus their total score in Rosetta energy unit (REU).

FIGS. 2E and 2F show in silico affinity maturation of the candidate designs. Both the total score and ddg are optimized.

FIGS. 2G and 2H show final designs (De1 and De3) bound to the ARD. The ARD-designed protein interface near the hotspot residues are shown in a zoom-in view.

FIG. 3 shows the assessment of peptide folding in candidate scaffolds.

FIG. 4A shows the interaction of the designed proteins with TRPV1 channel. Co-localization of TRPV1-YFP and De1-CFP in HEK293 cells revealed by Airyscan super resolution imaging. TRPV1-YFP and De1-CFP are pseudo-colored in red and green, respectively.

FIG. 4B shows FRET imaging of TRPV1-YFP and De1-CFP co-expressed in HEK293 cells. The emission spectra measured from the edge of cell (dotted arrows) are used for FRET efficiency calculation.

FIG. 4C shows the measured FRET ratio values for cells co-expressing the fluorophore-tagged TRPV1 and designed protein De1. The line indicates the predicted FRET Ratio values.

FIG. 4D shows the measured FRET ratio values for cells co-expressing the fluorophore-tagged TRPV1 and designed protein De2. The line indicates the predicted FRET Ratio values.

FIG. 4E shows the measured FRET ratio values for cells co-expressing the fluorophore-tagged TRPV1 and designed protein De3. The line indicates the predicted FRET Ratio values.

FIG. 4F shows the measured FRET ratio values for cells co-expressing the fluorophore-tagged TRPV1 and designed protein De4. The line indicates the predicted FRET Ratio values.

FIG. 4G shows the FRET efficiency measured from cells coexpressing the fluorophore-tagged TRPV1 and designed protein De1. The efficiency value was plotted as a function of the fluorescence intensity ratio between CFP and YFP. Each symbol represents a single cell. The solid curve represents the FRET model that yields the best fit; dotted curves represent models with 5% higher or lower FRET efficiencies.

FIG. 4H shows the FRET efficiency measured from cells coexpressing the fluorophore-tagged TRPV1 and designed protein De2, as described for FIG. 4G.

FIG. 4I shows the FRET efficiency measured from cells coexpressing the fluorophore-tagged TRPV1 and designed protein De3, as described for FIG. 4G.

FIG. 4J shows the FRET efficiency measured from cells coexpressing the fluorophore-tagged TRPV1 and designed protein De4 (closed circles), as described for FIG. 4G. FRET efficiency values from coexpression of CFP and YFP (open boxes) are overlaid.

FIG. 5 shows the expression of TRPV1 modulators De1 and De3.

FIG. 6A shows SPR signal traces of De1 at distinct concentration levels bound to the ARD fixed on the sensor chip, indicating binding of De1 to the ARD of TRPV1.

FIG. 6B shows SPR signal traces of De3 at distinct concentration levels bound to the ARD fixed on the sensor chip, indicating binding of De1 to the ARD of TRPV1.

FIG. 6C shows steady state concentration response curves of SPR signals to De1 binding to the ARD.

FIG. 6D shows steady state concentration response curves of SPR signals to De1 binding to the ARD.

FIG. 6E shows that no SPR signal was detected when De1 was first boiled to disrupt its three-dimensional structure.

FIG. 6F shows that no SPR signal was detected when De3 was first boiled to disrupt its three-dimensional structure.

FIG. 7A shows representative calcium imaging of capsaicin activation of TRPV1 alone or co-expressed with the designed proteins, indicating that De1 and De3 selectively potentiate ligand activation of TRPV1. Scale bar: 40 μm. Cells are pseudo colored.

FIG. 7B shows the concentration dependence of capsaicin activation of TRPV1 alone or co-expressed with the designed proteins in calcium imaging experiments (n=14-to-19). All statistical data are given as mean±±s.e.m.

FIG. 7C shows the concentration response curves of capsaicin activation of TRPV1 alone or co-expressed with the designed proteins in whole-cell patch clamp recordings (n=4-to-7). All statistical data are given as mean±s.e.m.**:p<0.01 in t-test.

FIG. 7D shows the EC50 values of capsaicin activation of TRPV1 alone or co-expressed with the designed proteins in whole-cell patch clamp recordings (n=4-to-7). All statistical data are given as mean±s.e.m.**:p<0.01 in t-test.

FIG. 7E shows concentration response curves of 2-APB activation of TRPV2 alone or co-expressed with the designed proteins in whole-cell patch clamp recordings (n=4-to-7). All statistical data are given as mean±s.e.m.

FIG. 7F shows concentration response curves of 2-APB activation of TRPV3 alone or co-expressed with the designed proteins in whole-cell patch clamp recordings (n=4-to-7). All statistical data are given as mean±s.e.m.

FIG. 8A shows current traces of capsaicin activation and acute desensitization of TRPV1 alone (bottom traces) or co-expressed with De3 (top traces) in the presence of extracellular calcium (1 mM), indicating that De3 alleviates acute desensitization and tachyphylaxis in ligand activation of TRPV1.

FIG. 8B shows time constants of acute desensitization of TRPV1 alone or co-expressed with designed proteins. *and ***:p<0.05 andp<0.001 in t-test, respectively, N.S., no significance in f-tests.

FIG. 8C shows representative current recordings of capsaicin induced tachyphylaxis of TRPV1 alone.

FIG. 8D shows representative current recordings of capsaicin induced tachyphylaxis of TRPV1 co-expressed with the designed protein De1.

FIG. 8E shows representative current recordings of capsaicin induced tachyphylaxis of TRPV1 co-expressed with the designed protein De3.

FIG. 8F shows measurements of tachyphylaxis as the current response amplitudes of the repeated capsaicin applications normalized to the first application. *:p<0.05 in t-test. All statistical data are given as mean±s.e.m.

FIG. 9A shows the binding of TAT-De3 to TRPV1 ARD as assessed by SPR.

FIG. 9B demonstrates the integrity of cell membranes after treatment with TAT-De3.

FIG. 10A shows representative whole-cell current recordings of capsaicin activation and acute desensitization of TRPV1 alone (grey) or with TAT-De3 (1 μM) perfused extracellularly (red) in the presence of extracellular calcium (1 mM).

FIG. 10B shows time constants of acute desensitization of TRPV1 alone or with TAT-De3 perfused extracellularly. *:p<0.05 in t-test. All statistical data are given as mean±s.e.m.

FIG. 10C shows changes in normalized paw withdrawal latency in rats injected with different ligands in heat pain tests. Paw withdrawal latency is normalized to the value measured before injection (n=6). *:p<0.05 in t-test. All statistical data are given as mean±s.e.m.

FIG. 10D shows body temperature of rats measured in the course of heat pain tests. N.S., no significance in two-way ANOVA tests (n=3-to-6). All statistical data are given as mean±s.e.m. Taken together, the data show that TAT-De3 exerts analgesic effects against heat pain in rats.

FIG. 10E shows a cartoon diagram illustrating the mechanisms underlying positive allosteric modulation of TRPV1 and analgesia.

### DETAILED DESCRIPTION OF THE INVENTION

Transient Receptor Potential Vanilloid 1 (TRPV1) ion channel is a nociceptor critically involved in pain sensation. Direct blockade of TRPV1 exhibits significant analgesic effects but also incurs severe side-effects such as hyperthermia, causing failures of TRPV1 inhibitors in clinical trials. In order to selectively target TRPV1 channels that are involved in pain-sensing, we de novo designed peptidic positive allosteric modulators (PAMs) based on the high-resolution structure of the TRPV1 intracellular ankyrin-repeat like domain. Our improved computational design method increased the success rate in protein binder design as compared to previous methods. We demonstrated experimentally, with a combination of FRET imaging, surface plasma resonance and patch-clamp recording, that the designed PAMs bind to TRPV1 with nanomolar affinity and allosterically enhance its response to ligand activation as we designed. We further demonstrated that the designed PAM exhibits long-lasting in vivo analgesic effects in rats without changing their body temperature, suggesting that they have potentials for developing into novel analgesics.

### I. Definitions

The terms “peptide,” “polypeptide,” and “protein” are used interchangeably herein to refer to a polymer of amino acid residues linked by covalent peptide bonds. All three terms apply to naturally occurring amino acid polymers and non-natural amino acid polymers, as well as to amino acid polymers in which one (or more) amino acid residue is an artificial chemical mimetic of a corresponding naturally occurring amino acid. Unless otherwise specified, the terms encompass amino acid chains of any length, including full-length proteins.

The term “amino acid” refers to any monomeric unit that can be incorporated into a peptide, polypeptide, or protein. Amino acids include naturally-occurring α-amino acids and their stereoisomers, as well as unnatural (non-naturally occurring) amino acids and their stereoisomers. “Stereoisomers” of a given amino acid refer to isomers having the same molecular formula and intramolecular bonds but different three-dimensional arrangements of bonds and atoms (e.g., an L-amino acid and the corresponding D-amino acid).

Naturally-occurring amino acids are those encoded by the genetic code, as well as those amino acids that are later modified, e.g. hydroxyproline, γ-carboxyglutamate and 0-phosphoserine. Naturally-occurring α-amino acids include, without limitation, alanine (Ala), cysteine (Cys), aspartic acid (Asp), glutamic acid (Gln), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gin), serine (Ser), threonine (Thr), valine (Val), tryptophan (Trp), tyrosine (Tyr), and combinations thereof. Stereoisomers of a naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D)-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” can be unnatural amino acids that have the same basic chemical structure as naturally-occurring amino acids (i.e., a carbon that is bonded to a hydrogen, a carboxyl group, an amino group) but have modified side-chain groups or modified peptide backbones, e.g., homoserine, norleucine, methionine sulfoxide, methionine methyl sulfonium. “Amino acid mimetics” refer to chemical compounds that have a structure that is different from the general chemical structure of an amino acid, but that functions in a manner similar to a naturally-occurring amino acid.

A “hotspot” amino acid residue refers to a residue characterized by energetically favorable interactions (including but not limited to hydrogen bonding, tight van der Waals packing, and complementary electrostatics) with a target region of a binding partner such as an TRPV1 ankyrin repeat domain.

The terms “nucleic acid,” “nucleotide,” and “polynucleotide” refer to deoxyribonucleic acids (DNA) or ribonucleic acids (RNA) and polymers. The term includes, but is not limited to, single-, double-, or multi-stranded DNA or RNA, genomic DNA, cDNA, and DNA-RNA hybrids, as well as other polymers comprising purine and/or pyrimidine bases or other natural, chemically modified, biochemically modified, non-natural, synthetic, or derivatized nucleotide bases. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), orthologs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al, Mol. Cell. Probes 8:91-98 (1994)).

“Percentage of sequence identity” is determined by comparing two optimally aligned sequences over a comparison window, wherein the portion of the sequence (e.g., a peptide of the invention) in the comparison window may comprise additions or deletions (i.e., gaps) as compared to the reference sequence which does not comprise additions or deletions, for optimal alignment of the two sequences. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity.

“Identical” and “identity,” in the context of two or more nucleic acids or polypeptide sequences, refer to two or more sequences or subsequences that are the same. Sequences are “substantially identical” to each other if they have a specified percentage of nucleotides or amino acid residues that are the same (e.g., at least 75%, at least 80%, at least 85%, at least 90%, at least 95%, or at least 99% identical over a specified region), when compared and aligned for maximum correspondence over a comparison window, or designated region as measured using one of the following sequence comparison algorithms or by manual alignment and visual inspection. These definitions also refer to the complement of a nucleic acid test sequence.

“Similarity” and “percent similarity,” in the context of two or more polypeptide sequences, refer to two or more sequences or subsequences that have a specified percentage of amino acid residues that are either the same or similar as defined by a conservative amino acid substitutions (e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% similar over a specified region), when compared and aligned for maximum correspondence over a comparison window, or designated region as measured using one of the following sequence comparison algorithms or by manual alignment and visual inspection. Sequences are “substantially similar” to each other if, for example, they are at least 20%, at least 25%, at least 30%, at least 35%, at least 40%, at least 45%, at least 50%, or at least 55% similar to each other.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Examples of algorithms that are suitable for determining percent sequence identity and sequence similarity are the BLAST and BLAST 2.0 algorithms, which are described in Altschul et al., (1990) J. Mol. Biol. 215: 403-410 and Altschul et al. (1977) Nucleic Acids Res. 25: 3389-3402, respectively. Software for performing BLAST analyses is publicly available at the National Center for Biotechnology Information website, ncbi.nlm.nih.gov. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see, e.g., Henikoff and Hetnikoff, Proc. Natl. Acad. Sci. USA 89:10915 (1989)).

The BLAST algorithm also performs a statistical analysis of the similarity between two sequences (see, e.g., Karlin and Altschul, Proc. Nat'l. Acad. Sci. USA, 90: 5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

As used herein the term “physiological conditions” refers to conditions that substantially replicate or otherwise approximate conditions inside an animal (e.g., a human). Exemplary physiological conditions include, but are not limited to, one or more parameters selected from 35-40° C., pH 7.35 to 7.45, osmolarity 250-350 mosM (e.g., 280-295 mosM), atmospheric pressure.

As used herein, the term “cell penetration peptide” refers to an amino acid sequence that, when linked to a second peptide (e.g., a TRPV1 modulator), causes or enhances the ability of the second peptide to cross the cell membrane of a cell when the cell is contacted by the cell penetration peptide linked to the second peptide.

As used herein, the term “pain” refers to the basic bodily sensation induced by a noxious stimulus, received by naked nerve endings, characterized by physical discomfort (e.g., pricking, throbbing, aching, etc.) and typically leading to an evasive action by the individual. As used herein, the term pain also includes chronic and acute neuropathic pain. Also included within the definition of pain include inflammatory pain (pain as the result of the inflammation, e.g., inflammatory hyperalgesia such as arthritis) and “other pain” (e.g., cancer pain, muscle pain, and headache).

As used herein, the terms “pruritus” and “itch” refer to an unpleasant sensation that evokes the desire or reflex to scratch. Itches are a common problem and can be localized (limited to one area of the body) or generalized (occurring all over the body or in several different areas). The medical term for itching is pruritus. Generalized itch, for obvious reasons, is more difficult to treat than localized itch. Itches can also occur with or without skin lesions (for example, bumps, blisters, or psoriasis).

As used herein, the term “cancer” refers to a neoplasm or tumor resulting from abnormal uncontrolled growth of cells. The term “cancer” encompasses disease states involving pre-malignant and/or malignant cancer cells. The cancer may be a localized overgrowth of cells that has not spread to other parts of a subject (e.g., a benign tumor), or the cancer may be have reached varying stages of invasion/destruction of neighboring body structures and spreading to distant sites (e.g., a malignant tumor).

As used herein, the terms “treat,” “treatment,” and “treating” refer to any indicia of success in the treatment or amelioration of an injury, pathology, condition, or symptom (e.g., lung cancer), including any objective or subjective parameter such as abatement; remission; diminishing of symptoms or making the symptom, injury, pathology or condition more tolerable to the patient; reduction in the rate of symptom progression; decreasing the frequency or duration of the symptom or condition; or, in some situations, preventing the onset of the symptom. The treatment or amelioration of symptoms can be based on any objective or subjective parameter, including, e.g., the result of a physical examination.

As used herein, the terms “effective amount” and “therapeutically effective amount” refer to a dose of a compound such as a cyclic dinucleotide that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); Goodman & Gillman's The Pharmacological Basis of Therapeutics, 11^{th} Edition, 2006, Brunton, Ed., McGraw-Hill; and Remington: the Science and Practice of Pharmacy, 21^{st} Edition, 2005, Hendrickson, Ed., Lippincott, Williams & Wilkins).

As used herein, the term “subject” refers to animals such as mammals, including, but not limited to, primates (e.g., humans), cows, sheep, goats, horses, dogs, cats, rabbits, rats, mice and the like.

As used herein, the term “pharmaceutically acceptable excipient” refers to a substance that aids the administration of an active agent to a subject. By “pharmaceutically acceptable,” it is meant that the excipient is compatible with the other ingredients of the formulation and is not deleterious to the recipient thereof. Pharmaceutical excipients useful in the compositions include, but are not limited to, binders, fillers, disintegrants, lubricants, glidants, coatings, sweeteners, flavors and colors.

As used herein, the terms “about” and “around” indicate a close range around a numerical value when used to modify that specific value. If “X” were the value, for example, “about X” or “around K” would indicate a value from 0.9X to 1.1X, e.g., a value from 0.95X to 1.05X, or a value from 0.98X to 1.02X, or a value from 0.99X to 1.01X. Any reference to “about X” or “around K” specifically indicates at least the values X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, and 1.1X, and values within this range.

### II. Peptidic Allosteric Modulators of TRPV1

The present invention was developed, in part, with the hypothesis that instead of using agonists or antagonists to modulate TRPV1 activity universally, positive allosteric modulators (PAMs) that selectively modulate high-activity population of TRPV1 is a promising alternative. TRPV1 channel is a calcium permeable channel highly expressed in nociceptive nerve termini (2). Previous studies have established that positive allosteric modulation of TRPV1 activities leads to local calcium overload in nociceptive afferent nerve terminus, causing functional and reversible inactivation of the nerve terminus to exert analgesic effects. For instance, the small molecule PAM of TRPV1 MRS1477, which was discovered in structure-activity relationship studies (10), enhances TRPV1 activation in the presence of orthosteric agonists such as capsaicin and exerts analgesic effects (11, 12).

To develop effective PAMs of TRPV1, instead of performing resource-consuming screening campaigns, we used a peptidic PAM design approach. Our method took advantage of the rich information from structural and functional investigations in TRPV1 (13-15) and rapidly evolving computational protein design using Rosetta (16, 17). Specifically, we chose the hotspot centric Rosetta computational approach which has been employed for the rational design of protein binders to specific domains of targets where three-dimensional structures have been resolved (18). Using this approach, previous studies reported successful de novo design of proteins bound to the stem region of hemaglutinin and the Fe domain of IgG (17, 19). Previous electrophysiological studies have shown that the ankyrin repeat-like domain (ARID) is critically involved in ligand induced desensitization of TRPV1 channel (13, 20, 21). We first established an improved protein design strategy to increase the success rate of obtaining robust designed binders. We then applied the improved Rosetta protein design approach to precisely target the ARD of TRPV1 to achieve positive allosteric modulation. Moreover, both the crystal structure of the ARD) and the cryo-EM structures of TRPV1 have been determined (13-15, 22). With a combination of our design method, FRET imaging, protein chemistry, surface plasma resonance, patch-clamp recordings and animal behavioral tests, we observed that two out of three of our designed PAMs bind to the ARD of TRPV1 with about 30 nano-molar affinity to positively modulate this channel in cells. Furthermore, we demonstrated that in rats our PAM exerts longer lasting analgesics effects as compared to MRS1477 without inducing hyperthemia.

Accordingly, some embodiments of the present disclosure provide A TRPV1 allosteric modulator comprising a peptide containing two hotspot amino acid residues,
wherein the hotspot amino acid residues are aromatic amino acid residues that interact with a TRPV1 ankyrin repeat domain (ARD), and
wherein alpha carbon atoms in the two hotspot amino acid residues are within 5-10 Å of each other upon folding of the peptide (e.g., under physiological conditions).

In some embodiments, the TRPV1 allosteric modulators prepared by process comprising:
i) assessing shape complementarity, binding energy, and solvent accessible surface area of a candidate peptide upon docking to a surface of a TRPV1 ankyrin repeat domain (ARD) in silico;
ii) assessing ab initio refolding of the candidate peptide;
iii) evolving the candidate peptide via in silico affinity maturation to define a mature allosteric modulator;
iv) expressing or synthesizing the mature allosteric modulator, thereby preparing the TRPV1 allosteric modulator.

In some embodiments, the process further comprises:
i-a) docking a surface of a peptide scaffold to a surface of the ARD to define a surface interface;
wherein the peptide scaffold comprises one or more ARD-binding hotspot amino acid residues and a plurality of non-hotspot amino acid residues;
i-b) fixing the hotspot amino acid residues to a target location on the surface of the TRPV1 ARD;
i-c) substituting at least one non-hotspot residue with another amino acid residue to define the candidate peptide.

In some embodiments, the target location on the surface of the TRPV1 ARD is located between two fingers of the ARD. For example, the target location on the surface of the TRPV1 ARD is located on the concave surface formed by the 2nd and 3rd fingers of the ARD. In some embodiments, the target location is located within the pocket formed by residues Y195, L206, I208, E211, F236, F246, and F248. For reference, the sequence of ARD domain (residues 112-359 of TRPV1) is set forth in SEQ TD NO:40.

Scaffold sequences can be designed and assessed for positioning of hotspot residues so as to promote or prevent particular interactions (e.g., binding events) with the target locations. For example, two phenylalanine hotspot residues may be positioned in an allosteric modulator such that the two alpha carbon atoms are within 5-10 Å of each other (e.g., about 7.98 Å in the case of De1 described below, or 6.27 Å in the case of De3 described below) as assessed in silico.

In some embodiments, the peptide scaffold is characterized by one or more properties selected from the group consisting of:
an X-ray crystal structure determined at a resolution of at least 2.5 Å;
an absence of DNA, RNA, or disulfide bonds in the X-ray crystal structure;
an absence of ligands or cofactors in the X-ray crystal structure;
no more than 200 amino acid residues in the primary amino acid sequence (e.g., no more than 150 amino acids, no more than 100 amino acids, or no more than 75 amino acids);
no more than one polypeptide chain;
monomeric stoichiometry; and
previous expression in E. coli reported.

Examples of peptide scaffolds include, but are not limited to, those listed in Table 1 below.

TABLE 1

| PDB ID | Wild-type Protein | Sequence | SEQ ID NO: |
|---|---|---|---|
| 1QZM | Alpha domain of Lon protease | SGYTEDEKLNIAKRHLLPKQIERN | 1 |
| | | ALKKGELTVDDSALIGURYYTRE | |
| | | AGVRGLEREISKLCRKAVKQLLL | |
| | | DKSLKHIEINGDNLHDYLGVQRF | |
| 3ONJ | Habc domain of SNAREs | SLLISYESDFKTTLEQAKASLAEA | 2 |
| | | PSQPLSQRNTTLKHVEQQQDELF | |
| | | DLLDQMDVEVNNSIGDASERATY | |
| | | KAKLREWKKTIQSDIKRPLQSLV | |
| | | DSGD | |
| 1XWC | Reduced Thioredoxin | MVYQVKDKADLDGQLTKASGKL | 3 |
| | | VVLDFFATWCGPCKMISPKLVEL | |
| | | STQFADNVVVLKVDVDECEDIA | |
| | | MEYNISSMPTFVFLKNGVKVEEF | |
| | | AGANAKRLEDVIKANI | |
| 2YXY | Hypothetical DUF1811-family | GHMIKGEQKRYSEMTKEELQQEI | 4 |
| | protein | AMLTEKARKAEQMGMVNEYAV | |
| | | YERKIAMAKAYMLNPADFHPGEI | |
| | | YEIEGAPGEYFKVRYLKGVFAWG | |
| | | WRLKGNGEEEALPISLLRKPNLPQS | |
| 1GYV | gama-Adaptin Appendage | MIPSITAYSKNGLKIEFTFERSNTN | 5 |
| | Domain | PSVTVITIQASNSTELDMTDFVFQ | |
| | | AAVPKTFQLQELSPSSSVVPAFNT | |
| | | GTITQVIKVLNPQKQQLRMRIKLT | |
| | | YNHKGSAMQDLAEVNNFPPQSWQ | |
| 3NGP | Redesigned alpha-spectrin SH- | MDETGKELVLVLYDYQEKSPREL | 6 |
| | 3 domain | TVKKGDILTLLNSTNKDWWKIEV | |
| | | NGRQGFVPAAYLKKLD | |
| 2HDZ | Upstream binding factor HMG | MGKLPESPKRAEEIWQQSVIGDY | 7 |
| | box5 | LARFKNDRVKALKAMEMTWNN | |
| | | MEKKEKLMWIKKAAEDQKRYER | |
| | | ELSEMRAPPAATNSSKKLE | |

In some embodiments:
the peptide scaffold and/or the TRPV1 allosteric modulator comprises a helix¹-loop-strand¹-helix²-turn-helix³-strand²-helix⁴ architecture,
strand¹ and strand² and folded in a parallel β sheet, and
the hotspot amino acid residues are phenylalanine residues located in helix² and helix⁴. The hotspot residues may be located, for example, at position 36 and position 83 of SEQ ID NO:8.

In some embodiments, the TRPV1 allosteric modulator includes a polypeptide sequence having at least 70% identity (e.g., at least 70%, 75%, 80%, 85%, 90%, 95%, or 99% identity) to positions 36-83 of SEQ ID NO:8, or 36-85 of SEQ ID NO:8, or positions 36-87 of SEQ ID NO 8, or 35-89 of SEQ ID NO:8, provided that the amino acid residues at position 36 and position 83 are aromatic amino acid residues.

In some embodiments, the TRPV1 allosteric modulator includes a polypeptide sequence having at least 70% identity (e.g., at least 70%, 75%, 80%, 85%, 90%, 95%, or 99% identity) to positions 35-89 SEQ ID NO:8 provided that the amino acid residues at position 36 and position 83 are aromatic amino acid residues (e.g., phenylalanine residues).

In some embodiments, the TRPV1 allosteric modulator includes a polypeptide sequence having at least 70% identity (e.g., at least 70%, 75%, 80%, 85%, 90%, 95%, or 99% identity) to SEQ ID NO:8. In some embodiments, the TRPV1 allosteric modulator includes a polypeptide sequence having at least 70% identity (e.g., at least 70%, 75%, 80%, 85%, 90%, 95%, or 99% identity) to SEQ ID NO:8 provided that the amino acid residues at position 36 and position 83 are phenylalanine residues.

In some embodiments:
the peptide scaffold and/or the TRPV1 allosteric modulator comprises a helix¹-loop-helix²-loop-helix³ architecture,
helix¹, helix², and helix³ are folded in a three-helix bundle, and
the hotspot amino acid residues are phenylalanine residues located in helix³. The hotspot residues may be located, for example, at position 72 and position 76 of SEQ ID NO:9.

In some embodiments, the TRPV1 allosteric modulator includes a polypeptide sequence having at least 70% identity (e.g., at least 70%, 75%, 80%, 85%, 90%, 95%, or 99% identity) to positions 70-78 of SEQ ID NO:9, or positions 68-80 of SEQ ID NO:9, or positions 66-82 of SEQ ID NO:9, or positions 64-84 of SEQ ID NO:9, or positions 64-86 of SEQ ID NO:9, or positions 64-90 of SEQ ID NO:9, positions 64-94 of SEQ ID NO-9, provided that the amino acid residues at position 72 and position 76 are aromatic amino acid residues (e.g., phenylalanine residues).

In some embodiments, the TRPV1 allosteric modulator includes a polypeptide sequence having at least 70% identity (e.g., at least 70%, 75%, 80%, 85%, 90%. 95%, or 99% identity) to positions 64-94 of SEQ ID NO:9. In some embodiments, the TRPV1 allosteric modulator includes a polypeptide sequence having at least 70% identity (e.g., at least 70%, 75%, 80%, 85%, 90%, 95%, or 99% identity) to positions 64-94 of SEQ ID NO:9 provided that the amino acid residues at position 72 and position 76 are aromatic amino acid residues (e.g., phenylalanine residues).

In some embodiments, the TRPV1 allosteric modulator includes a polypeptide sequence having at least 70% identity (e.g., at least 70%, 75%, 80%, 85%, 90%, 95%, or 99% identity) to SEQ ID NO:9. In some embodiments, the TRPV1 allosteric modulator includes a polypeptide sequence having at least 70% identity (e.g., at least 70%, 75%, 80%, 85%, 90%, 95%, or 99% identity) to SEQ ID NO:9 provided that the amino acid residues at position 72 and position 76 are phenylalanine residues.

Examples of designed peptides using, the scaffolds described herein include, but are not limited to, those set forth in fable 2.

TABLE 2

| | | | | Muta- | | | | |
|---|---|---|---|---|---|---|---|---|
| | | | | tions | | | | |
| | | | | from | | | | |
| Design | | | No. | wild- | | | | |
| candidate | Scaf- | | of | type | | | | Shape |
| (SEQ ID | fold | Wild-type | Resi- | Scaffold | Total | | | comple- |
| NO) | PDB | function | dues | sequence | score | Ddg | SASA | mentarity |
| 15237 | 1QZM | Alpha | 93 | 23 | −497.304 | −27.703 | 1929.842 | 0.6743 |
| (SEQ | | domain of | | | | | | |
| ID NO: | | Lon | | | | | | |
| 10) | | protease | | | | | | |
| 15237_ | 3ONJ | Habc | 96 | 27 | −531.014 | −31.511 | 2757.631 | 0.5324 |
| 2nd_0086 | | domain of | | | | | | |
| (SEQ | | SNAREs | | | | | | |
| ID NO: | | | | | | | | |
| 11) | | | | | | | | |
| 5132 | 3ONJ | Habc | 96 | 20 | −480.546 | −24.915 | 2450.968 | |
| (SEQ ID | | domain of | | | | | | |
| NO: 12) | | SNAREs | | | | | | |
| 11577 | 1XWC | Reduced | 105 | 32 | −494.240 | −31.551 | 2512.314 | 0.6044 |
| SEQ ID | | Thiore- | | | | | | |
| NO: 13) | | doxin | | | | | | |
| 2824 | 2YXY | Hypo- | 103 | 31 | −560.978 | −30.506 | 3086.675 | 0.6102 |
| (SEQ ID | | thetical | | | | | | |
| NO: 14) | | | | | | | | |
| 926 | 1GYV | gama- | 119 | 32 | −560.790 | −26.620 | 2352.198 | 0.6016 |
| (SEQ ID | | Adaptin | | | | | | |
| NO: 15) | | Appendage | | | | | | |
| | | Domain | | | | | | |
| 15080 | 3NGP | Redesigned | 56 | 23 | −397.464 | −27.201 | 1772.034 | 0.6287 |
| SEQ ID | | SH-3 | | | | | | |
| NO: 16) | | domain | | | | | | |
| 15080_cst | 3NGP | | 56 | 21 | −402.347 | −22.370 | 1909.638 | |
| SEQ ID | | | | | | | | |
| NO: 17) | | | | | | | | |
| 8320_ | 2HDZ | upstream | 65 | 12 | −444.04 | −15.870 | 1527.721 | |
| 2nd_0141 | | binding | | | | | | |
| (SEQ ID | | factor | | | | | | |
| NO: 18) | | HMG | | | | | | |
| | | box 5 | | | | | | |

In some embodiments, the TRPV1 allosteric modulator may contain one or more amino acid substitutions, deletions, or additions with respect to a given sequence (e.g., a scaffold sequence used as a starting point for modulator development, or an intermediate sequence obtained during computation design as described herein). Certain substitutions will be recognized as “conservative” modification where the alteration results in the substitution of an amino acid with a chemically similar amino acid. Chemically similar amino acids may include, without limitation, genetically-encoded amino acids such as an L-amino acids, stereoisomers of genetically-encoded amino acids such as a D-amino acids, N-substituted amino acids (e.g., n-methylglycine), amino acid analogs, amino acid mimetics, and synthetic amino acids. Conservative substitution tables providing functionally similar amino acids are well known in the art. For example, substitutions may be made wherein an aliphatic amino acid (e.g., G, A, I, L, or V) is substituted with another member of the group. Similarly, an aliphatic polar-uncharged group such as C, S, T, M, N. or Q, may be substituted with another member of the group; and basic residues, e.g., K, R, or H, may be substituted for one another. In some embodiments, an amino acid with an acidic side chain, e.g., E or D, may be substituted with its uncharged counterpart, e.g., Q or N, respectively; or vice versa. Each of the following eight groups contains other exemplary amino acids that are conservative substitutions for one another (see, e.g., Creighton, Proteins, 1993):
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)

ARD target peptides and modulator scaffold peptides, with or without amino acid mutations, may be assessed using on or modeling steps as described herein. ARD target peptides may be relaxed, for example, by executing a script such as Script 1 as set forth below.

Script 1

#!/bin/bash
#$ -S /bin/bash
#$ -e /home/fanyang/work/constrained_relax_2PNN-2PNN-_/$TASK_ID/
#$ -o /home/fanyang/work/constrained_relax_2PNN-2PNN-_/$TASK_ID/
/home/fanyang/rosetta_source/bin/relax. 1inuxgccrelease \
-in:path:database /home/fanyang/rosetta_database \
-in:file:s /home/fanyang/projects/input_files/2PNN/2PNN_cleaned_fy. pdb \
-ignore_unrecognized_res \
-relax:fast \
-relax:constrain_relax_to_start_coords \
-relax:coord_constrain_sidechains \
-relax:ramp_constraints false \
-ex1 \
-ex2 \
-use_input_sc \
-correct \
-nstruct 100 \
-out:prefix constrained-relax- \
-out:file:silent /home/fanyang/work/constrained_relax_2PNN-2PNN-
_/${SGE_TASK_ID} /constrained-relax-_2PNN_${SGE_TASK_ID}.silent \
-out:file:silent_struct_type binary \
-mute all

Inverse rotamer libraries of candidate hotspot residues may be constructed by executing a script such as Script 2 as shown below.

Script 2

```
-database /home/fan/rosetta_source/rosetta_database
-ignore_unrecognized_res
-in:file:fullatom
-ex1
-ex2
-in:file:s
/media/Data2/Academic/Rosetta/Project_Ank/HS_Candidates/PheHS2_PheHS1_0510/Rosetta
Dock_Phe_ini_Phe_HS1_2PNN_CCR_0077_3730. pdb
-parser:protocol
/media/Data2/Academic/Rosetta/Rosetta_script/xml_scripts_Fan/InverseRotamers_fy. xm
l
-nstruct 1000
-overwrite
InverseRotamers_fy. xml :
<dock_design>
  <SCOREFXNS>
    <stub_dock weights=talaris2014 />
  </SCOREFXNS>
  <FILTERS>
    <EnergyPerResidue name=energy scorefxn=stub_dock pdb_num=1B
energy_cutoff=1/>
    <Ddg name=ddg scorefxn=stub_dock threshold =−1 repack=0/>
  </FILTERS>
  <MOVERS>
    <TryRotamers name=try pdb_num=1B /> list residues the backbones of
which are to be ignored in energy evaluations under the shove flag
    RepackMinimize name=rpk repack_partner1=1 repack_partner2=0
design_partner1=0 design_partner2=0 minimize bb=0 minimize_rb=0 minimize sc=1
  </MOVERS>
  <APPLY_TO_POSE>
  </APPLY_TO_POSE>
  <PROTOCOLS>
    <Add mover_name=try/>
    add mover_name=rpk
    <Add filter_name=energy/>
    <Add filter_name=ddg/>
  </PROTOCOLS>
</dock_design>
```

Scaffold candidate structures may be cleaned by executing a script such as Script 3 as shown below.

Script 3

```
#!/bin/bash
FOLDER_A=/media/Data_Ubuntu/PatchDock/scaffold_0402
FOLDER_B=/media/Data_Ubuntu/PatchDock/scaffold_cleaned_0402
for file_a in ${FOLDER_A}/*; do
  file_a_prefix=${file_a%. *}
  file_prefix=${file_a_prefix#${FOLDER_A} /*}
  echo $file_prefix
  sudo cleanPdb. p1 -pdbfile ${FOLDER_A} /$file_prefix. pdb >
$ {FOLDER_B}/$file_prefix. pdb
done
```

Cleaned scaffold candidate structures may be pre-packed by executing a script such as Script 4 as shown below.

Script 4

```
#!/bin/bash
FOLDER_A=/media/Data_Ubuntu/PatchDock/scaffold_0405_cleaned
for file_a in ${FOLDER_A}/*; do
  file_a_prefix=${file_a%.*}
  file_prefix=${file_a_prefix#${FOLDER_A}/*)
  echo $file_prefix
  /home/fan/rosetta_source/bin/rosetta_scripts. 1inuxgccrelease -ex1 -ex2aro -
database /home/fan/rosetta_source/rosetta_database -ignore_unrecognized_res -
in:file:fullatom -in:file:s
/home/fan/PatchDock/PDB/scaffold_PDB_1/$file_prefix. pdb -parser:protocol
/home/fan/rosetta_source/input_files/prepack_2PNN/ppk. xml -overwrite -out:output
  sudo my $file_prefix*. pdb $file_prefix. pdb
  sudo cp $file_prefix.pdb
/media/Data_Ubuntu/PatchDock/scaffold_0405_cleaned_prepacked/
  rm -r $file_prefix. pdb
done
```

Scaffold proteins may be fused with hotspots by executing a script such as Script 5 as shown below.

Script 5

```
#!/bin/bash
#$ -S /bin/bash
#$ -e /home/fanyang/work/Placestub_PheHS2_PheHS1_2PNN_0511-2PNN-_/$TASK_ID/
#$ -o /home/fanyang/work/Placestub_PheHS2_PheHS1_2PNN_0511-2PNN-_/$TASK_ID/
n=(${SGE_TASK_ID}-1)*100+1
for ((i=1; i<101; i=i+1))
do /home/fanyang/rosetta_source/bin/rosetta_scripts. static. 1inuxgccrelease \
-in:path:database /home/fanyang/rosetta_database \
-in:file:fullatom \
-ex1 \
-ex2 \
-in:file:s /home/fanyang/projects/input_files/2PNN/Docked_0510/$[${i} +${n}]. pdb \
-out:file:scorefile mscore_$[${i} +${n}].fsc \
-nstruct 50 \
-out:file:silent /home/fanyang/work/Placestub_PheHS2_PheHS1_2PNN_0511-2PNN-
_/${SGE_TASK_ID}/twoHS_placestubs_2PNN_${SGE_TASK_ID} . silent \
-out:file:silent_struct_type binary \
-mute all \
-parser:protocol
/home/fanyang/projects/input_files/2PNN/TwoResidueHotspot_fy_0511. xml
done
TwoResidueHotspot_fy_0511. xml:
<ROSETTASCRIPTS>
        <TASKOPERATIONS>
            <PreventRepacking name=prevent_repacking_Y84 resnum=84/>
            <PreventRepacking name=prevent_repacking_Y89 resnum=89/>
        </TASKOPERATIONS>
        <SCOREFXNS>
            <stub_docking_low weights=interchain_cen hs_hash=10. 0/>
            <ddg_scorefxn weights=standard patch=score12 hs_hash=0. 0/>
            <score12_coordest weights=standard patch=score12 hs_hash=0. 0>
                <Reweight scoretype=coordinate_constraint weight=1. 0/>
            </score12_coordest>
  </SCOREFXNS>
    <FILTERS>
              <Ddg name=ddg threshold=-10 scorefxn=ddg_scorefxn repeats=3/>
              <Sasa name=sasa threshold=1200/>
              <CompoundStatement name=ddg_sasa> for the loop over filter
                    <AND filter_name=ddg/>
                    <AND filter_name=sasa/>
                </CompoundStatement>
                <TerminusDistance name=termini distance=3/>
        <AtomicContact name=touch_125 residue1=125A distance=5/>
        <AtomicContact name=touch 89 residue1=89A distance=5/>
        <AtomicContact name=touch 84 residue1=84A distance=5/>
        <AtomicContact name=touch_53 residue1=53A distance=5/>
        <AtomicContact name=touch_97 residue1=97A distance=5/>
                <CompoundStatement name=touch_Trp>
                    <AND filter_name=touch_125/>
                    <AND filter_name=touch_89/>
                      <AND filter_name=touch_97/>
                </CompoundStatement>
                  <CompoundStatement name=touch_Leu>
                      <AND filter_name=touch_84/>
                      <AND filter_name=touch_53/>
                  </CompoundStatement>
    </FILTERS)
    <MOVERS>
      <Docking name=dock fullatom=0 local_refine=0 score_low=stub_docking_low/>
                <RepackMinimize name=des1 minimize_bb=0 minimize_rb=1
scorefxn_repack=soft_rep scorefxn_minimize=score_docking
interface_cutoff_distance=10. 0/>
                <RepackMinimize name=des2 minimize_bb=0 minimize_rb=1
interface_cutoff_distance=10. 0/>
                <RepackMinimize name=des3 minimize_bb=1 minimize_rb=1
interface_cutoff_distance=10. 0/>
                <SaveAndRetrieveSidechains name=srsc/>
                <BackrubDD name=br partner1=0 partner2=1
interface_distance_cutoff=10. 0/>
                <PlaceStub name=place top minimize_rb=1 triage_positions=1
stubfile=” /home/fanyang/projects/input_files/2PNN/HS_lib/PheHS2_IR. pdb”
stub_energy_threshold=10 final_filter=ddg_sasa hurry=1 max_cb dist=4.0
task_operations=prevent_repacking_Y84>
                    <StubMinimize>
                        <Add mover_name=br/>
                    </StubMinimize>
                    <DesignMovers>
                        <Add mover_name=srsc/>
                        <Add mover_name=des1 coord_est_std=1. 0/>
                        <Add mover_name=des2 coord_est_std=1. 5/>
                        <Add mover_name=br/>
                        <Add mover_name=des3/>
                    </DesignMovers>
                </PlaceStub>
                <PlaceStub name=place_trp place_scaffold=1 triage_positions=0
stubfile= ”/home/fanyang/projects/input_files/2PNN/HS_lib/PheHS1_IR. pdb”
task_operations=prevent_repacking_Y89 add_constraints=1 minimize_rb=1 hurry=1
score_threshold=10. 0 stub_energy_threshold=3. 0 max_cb_dist=4. 0 leave_coord_csts=1
task_operations=prevent_repacking_Y84> PlaceStub's task operations will be fed
down to all movers that are mentioned as task aware. leave_coord_ests lets
downstream movers use the coordinate ests that placestub found to be useful
                    <StubMinimize>
                        <Add mover_name=br/>
                    </StubMinimize>
                    <DesignMovers>
                        <Add mover_name=place_top coord_cst_std=1. 0/>
                    </DesignMovers>
                    </PlaceStub>
          </MOVERS>
      <APPLY_TO_POSE>
          <SetupHotspotConstraints
stubfile=” /home/fanyang/projects/input_files/2PNN/HS_lib/PheHS2_PheHS1_IR. pdb”
cb_force=0. 5/>
      </APPLY_TO_POSE>
      <PROTOCOLS>
                <Add mover_name=dock filter_name=termini/>
                    <Add mover_name=place_trp/>
                    <Add filter_name=ddg/>
                    <Add filter_name=sasa/>
      </PROTOCOLS>
</ROSETTASCRIPTS>
```

In silico affinity maturation nay be conducted by executing a script such as Script 6 as shown below.

Script 6

```
-database /home/fan/rosetta_source/rosetta_database
-ignore_unrecognized_res
-in:file:fullatom
-ex1
-ex2
-in:file:s /media/Data2/Academic/Rosetta/candidate. pdb
-parser:protocol
/media/Data2/Academic/Rosetta/Rosetta_script/xml_scripts_Fan/MulticriterionOptimiz
ation. xml
-nstruct 100
-overwrite
MulticriterionOptimization. xml:
<dock_design>
  <TASKOPERATIONS>
      <InitializeFromCommandline name=init/>
      <ProteinInterfaceDesign name=pido/>
    <RestrictResiduesToRepacking name=restrict_all/>
      <RestrictAbsentCanonicalAAS name=nohis
keep_aas=” ACDEFGIKLMNPQRSTVWY” />
  </TASKOPERATIONS>
  <SCOREFXNS>
  </SCOREFXNS>
  <FILTERS>
      <Sasa name=hydrophobic_sasa hydrophobic=1 confidence=0/>
      <Sasa name=sasa confidence=0/>
      <Ddg name=ddg confidence=0 repeats=3/>
      <ScoreType name=total_score score_type=total_score threshold=0/>
  </FILTERS>
  <MOVERS>
      <AtomTree name=docking_tree docking_ft=1/>
      <RandomMutation name=random_mutation
task_operations=init, pido, restrict_all, nohis/>
      <MinMover name=min bb=1 chi=1 jump=1>
        <MoveMap>
          <Chain number=1 chi=1 bb=0/>
        </MoveMap>
      </MinMover>
      <ParsedProtocol name=agg_mover>
        <Add mover=random_mutation/>
        <Add mover=min/>
      </ParsedProtocol>
      <GenericMonteCarlo name=genericMC mover_name=agg_mover
filter_name=ddg temperature=0. 1 trials=1000>
        <Filters>
          <AND filter_name=total score temperature=1/>
        </Filters>
      </GenericMonteCarlo>
  </MOVERS>
  <APPLY_TO_POSE>
  </APPLY_TO_POSE>
  <PROTOCOLS>
      <Add mover=docking_tree/>
      <Add mover=genericMC/>
      <Add filter=ddg/>
      <Add filter=sasa/>
  </PROTOCOLS>
</dock_design>
```

Any design candidate can be assessed for the ability to bind TRPV1 using techniques including, but not limited to, the FRET protocols and SPR protocols described herein. For example, complementary fluorescent proteins (including, but not limited to, GFP CFP, YFP, and the like) may be fused recombinantly to a designed peptide and to TRPV1 for assessment of TRPV1 binding via FRET. In some non-limiting embodiments, binding of a candidate modulator to TRPV1 can be confirmed by observing a FRET ratio max of at least 1.25 when YFP is fused to the C-terminus of TRPV1, CFP is fused to the C-termini of a candidate peptide. In some embodiments, binding of a candidate modulator to TRPV1 can be confirmed by observing a FRET efficiency greater than about 4% (e.g., at least 5%, or at least 7%) when YFP is fused to the C-terminus of TRPV1, CFP is fused to the C-termini of a candidate peptide. FRET measurements can be made by co-expressing the FRET-labeled constructs in host cells, exciting the CFP construct in the host cells at 458 inn, and observing YFP emission at 530 nm. CFP emission can be separated from YFP emission by fitting of standard spectra acquired from cells expressing YFP or CFP only.

FRET efficiency is defined in its usual definition; it is inversely related to the sixth power of the distance between complementary fluorescent proteins on the binding partners. FRET efficiency is quantified from measurements of the fluorescence emission spectra, using the established method. Judgement of positive FRET signals can be made by comparing them to that obtained from a mixture of complementary fluorescent proteins not expected to exhibit specific association (i.e., a negative control); signals significantly higher than that of a negative control indicate specific binding of a design candidate to TRPV1.

FRET ratios can be calculated from the increase in YFP emission due to energy transfer as described previously (Qiu, et al. J. Biol. Chem. 2005, 280, 24923, incorporated herein by reference in its entirety). CFP emission can be separated from YFP emission by fitting of standard spectra acquired from cells expressing only YFP or CFP. The fraction of YFP-tagged molecules that are associated with CFP-tagged molecules, Ab, is calculated as Ab=1/(1+K_{D}/[Dfree]), where K_{D} is the dissociation constant and [Dfree] is the concentration of free donor molecules. The FRET Ratio=1+Ab*(FRET Ratioₘₐₓ−1). Regression analysis can be used to estimate Ab in individual cells. From each cell, the FRET ratioₑₓₚ is experimentally determined. The predicted Ab value is then computed by adjusting two parameters, FRET Ratioₘₐₓ, and apparent K_{D} Ab was in turn used to give a predicted FRET ratio_{predicted}. By minimizing the squared errors (FRET ratioₑₓₚ. FRET ratio_{predicted})², K_{D} was determined.

Apparent FRET efficiency can be calculated from the enhancement of YFP acceptor fluorescence emission due to energy transfer from the CFP donor using previously described methods. [see, e.g., Yang and Zheng, et al. Proc. Natl. Acad. Sci. U.S.A. 2010, 107 7083, which is incorporated herein by reference]. RatioA₀, and RatioA₁, are measured to calculate FRET efficiency. RatioA₀, represents the ratio between acceptor emission intensities (in the absence of the donor) upon excitation at the donor and acceptor excitation wavelengths, and can be calculated at the YFP peak emission wavelength. A particular advantage of quantifying RatioA₀ for FRET measurement is that changes in fluorescence intensity caused by many experimental factors cap be cancelled out by the ratiometric measurement:

$${Ratio A}_{0} = \frac{{F}_{d}^{direct}}{{F}_{a}}$$

in which F_{d} and Fₐ denote fluorescence intensities measured upon excitation at the donor and acceptor excitation wavelength, respectively, F_{d}^{direct} represents direct excitation of YFP by excitation at the donor excitation wavelength. A similar ratio, termed RatioA, is determined in the presence of CFP in the same way as RatioA₀

$${Ratio A}_{0} = \frac{{F}_{d}^{direct}}{{F}_{a}} + \frac{{F}_{d}^{FRET}}{{F}_{a}}$$

in which F_{d}^{FRET} represents the fluorescence signal due to FRET.

If FRET occurs, the RatioA value should be higher than RatioA₀; the difference between RatioA and RatioA₀, is directly proportional to the FRET efficiency by the factor of extinction coefficient ratio of CFP and YFP. The effective FRET efficiency (E) can be calculated as:

$$E = \frac{{ε}_{YFP}}{{ε}_{CFP}} ( \frac{Ratio A}{Ratio {A}_{0}} - 1 )$$

In some embodiments, the TRPV1 allosteric modulator further comprises a cell penetration peptide sequence. The cell penetration peptide sequence may be linked, for example, to the N-terminus of the TRPV1 allosteric modulator or the C-terminus of the TRPV1 allosteric modulator). In some embodiments, the C-terminus of the cell penetration peptide is linked to the N-terminus of the TRPV1 allosteric modulator. A number of cell penetration peptides can be linked to the TRPV1 allosteric modulator so as to enhance delivery of the TRPV1 allosteric modulator to target cells in vitro and/or in vivo (see, e.g., Guidotti, et al. Trends in Pharmacological Sciences. 2017, 38(4): 406-424). In some embodiments, the cell penetration peptide is a cationic peptide having 5-25 total amino acid residues and at least 5 arginine residues, lysine residues, or a combination thereof. In some embodiments, the cell penetration peptide is a polyarginine ranging in length from 5 residues to 25 residues. In some embodiments, the cell penetration peptide comprises an amino acid sequence set forth in Table 3 or a sequence having at least 70% identity (e.g., at least 70%, 75%, 80%, 85%, 90% 95% or 99% identity) thereto.

TABLE 3
Exemplary Cell Penetration Peptides

| SEQ | | |
|---|---|---|
| ID | | |
| NO: | Sequence | Origin |
| 19 | RKKRRQRRR | HIV-1 Tat protein |
| 20 | YGRKKRRQRRRA | HIV-1 Tat protein |
| 21 | GRKKRRQRRRPPQ | HIV-1 Tat protein |
| 22 | RQIKIWFQNRRMKWKK | Antennapedia |
| | | Drosophila |
| | | melanogaster |
| 23 | VKRGLKLRHVRPRVTRMDV | Chemically |
| | | synthesized |
| 24 | GALFLGFLGAAGSTMGAWSQPKKKRKV | HIV glycoprotein |
| | | 41/SV40 T antigen |
| | | NLS |
| 25 | KETWWETWWTEWSQPKKKRKV | Tryptophan-rich |
| | | cluster/SV40 T |
| | | antigen NLS |
| 26 | LLIILRRRIRKQAHAHSK | Vascular |
| | | endothelial |
| | | cadherin |
| 27 | MVRRFLVTLRIRRACGPPRVRV | p14ARF protein |
| 28 | MVKSKIGSWILVLFVAMWSDVGLCKKRP | N terminus of |
| | | unprocessed bovine |
| | | prion protein |
| 29 | KLALKLALKALKAALKLA | Chemically |
| | | synthesized |
| 30 | GWTLNSAGYLLGKINLKALAALAKKIL | Chimeric galanin- |
| | | mastoparan |
| 31 | LSTAADMQGVVTDGMASGLDKDYLKPDD | Azurin |
| 32 | DPKGDPKGVTVTVTVTVTGKGDPKPD | Synthetic |
| 33 | RRIRPRPPRLPRPRPRPLPFPRPG | Bactenecin family |
| | | of antimicrobial |
| | | peptides |
| 34 | CSIPPEVKFNKPFVYLI | α1-Antitrypsin |
| 35 | PFVYLI | Derived from |
| | | synthetic C105Y |
| 36 | SDLWEMMMVSLACQY | CHL8 peptide |
| | | phage clone |

In some embodiments, the cell penetration peptide is an HIV-1 Tat peptide (e.g., a peptide according to SEQ ID NO: 19, 20, or 21). In some embodiments, the HIV-1 Tat peptide comprises an amino acid sequence having at least 70% identity to SEQ ID NO:19. In some embodiments, the HIV-1 Tat peptide comprises an amino acid sequence having at least 80% identity to SEQ ID NO: 19. In some embodiments, the HIV-1 Tat peptide comprises an amino acid sequence having at least 90% identity to SEQ ID NO:19. In some embodiments, the peptide construct comprises a cell penetration peptide having the amino acid sequence according to SEQ ID NO:19 and a TRPV1 allosteric modulator comprising an amino acid sequence having at least 70% identity (e.g., at least 80% identity, or at least 90% identity) to SEQ ID NO:8 or SEQ ID NO:9.

A cell penetration peptide may also be chemically modified, such as prenylated near the C-terminus of the cell penetration peptide. Prenylation is a post-translation modification resulting in the addition of a 15 (farnesyl) or 20 (geranylgeranyl) carbon isoprenoid chain on the peptide. A chemically modified cell penetration peptide can be even shorter and still possess the cell penetrating property.

Accordingly, a cell penetration peptide, pursuant to another aspect of the disclosure, is a chemically modified CPP with 2 to 35 amino acids, preferably 5 to 25 amino acids, more preferably 10 to 25 amino acids, or even more preferably 15 to 25 amino acids.

Cell penetration peptides can be linked to a protein recombinantly, covalently or non-covalently. A recombinant protein having a cell penetration peptide can be prepared in bacteria, such as E. coli, a mammalian cell such as a human HEK293 cell, or any cell suitable for protein expression. Covalent and non-covalent methods have also been developed to form CPP/protein complexes. A cell penetration peptide, Pep-1, has been shown to form a protein complex and proven effective for delivery (Kameyama et al. (2006) Bioconjugate Chem. 17:597-602).

Cell penetration peptides also include cationic conjugates which also may be used to facilitate delivery of the proteins into the cells or tissue of interest. Cationic conjugates may include a plurality of residues including amines, guanidines, amidines, N-containing heterocycles, or combinations thereof. In related embodiments, the cationic conjugate may comprise a plurality of reactive units selected from the group consisting of alpha-amino acids, beta-amino acids, gamma-amino acids, cationically functionalized monosaccharides, cationically functionalized ethylene glycols, ethylene imines, substituted ethylene imines, N-substituted spermine, N-substituted spermidine, and combinations thereof. The cationic conjugate also may be an oligomer including an oligopeptide, oligoamide, cationically functionalized oligoether, cationically functionalized oligosaccharide, oligoamine, oligoethyleneimine, and the like, as Nell as combinations thereof. The oligomers may be oligopeptides where amino acid residues of the oligopeptide are capable of forming positive charges. The oligopeptides may contain 5 to 25 amino acids; preferably 5 to 15 amino acids; more preferably 5 to 10 cationic amino acids or other cationic subunits.

In a related aspect, the present disclosure provides nucleic acids encoding peptide constructs as described herein. The nucleic acids can be generated from a nucleic acid template encoding a scaffold protein or a designed TRPV1 allosteric modulator, using any of a number of known recombinant DNA techniques. Accordingly, certain embodiments of the present disclosure provide an isolated nucleic acid comprising a polynucleotide sequence encoding a peptide construct comprising a TRPV1 allosteric modulator (including, but not limited to, peptide constructs comprising amino acid sequences having at least 70%, 80%, or 90% identity to SEQ ID NO:8 or SEQ ID NO:9 and optionally comprising cell penetration sequences such as SEQ ID NO: 19). Using these nucleic acids, a variety of expression constructs and vectors can be made. Generally, expression vectors include transcriptional and translational regulatory nucleic acid regions operably linked to the nucleic acid encoding the TRPV1 allosteric modulator. Control sequences that are suitable for prokaryotes, for example, include a promoter, optionally an operator sequence, and a ribosome binding site. In addition, the vector may contain a Positive Retroregulatory Element (PRE) to enhance the half-life of the transcribed mRNA (see, Gelfand et at. U.S. Pat. No. 4,666,848). The transcriptional and translational regulatory nucleic acid regions will generally be appropriate to the host cells used to express the peptide constructs. Numerous types of appropriate expression vectors and suitable regulatory sequences are known in the art for a variety of host cells. In general, the transcriptional and translational regulatory sequences may include, e.g. promoter sequences, ribosomal binding sites, transcriptional start and stop sequences, translational start and stop sequences, and enhancer or activator sequences. Typically, the regulatory sequences will include a promoter and/or transcriptional start and stop sequences. Vectors also typically include a polylinker region containing several restriction sites for insertion of foreign DNA. Heterologous sequences (e.g., a fusion tag such as a His tag) can be used to facilitate purification and, if desired, removed after purification. The construction of suitable vectors containing DNA encoding replication sequences, regulatory sequences, phenotypic selection genes, and TRPV1 allosteric modulators can be prepared using standard recombinant DNA procedures. Isolated plasmids, viral vectors, and DNA fragments can be cleaved, tailored, and ligated together in a specific order to generate the desired vectors, as is well-known in the art (see, e.g., Green and Sambrook, Molecular Cloning: A Laboratory Manual (Cold Spring Harbor Laboratory Press, New York, 4^{th} ed. 2012)).

Provided in some embodiments is an expression cassette comprising a nucleic acid encoding a TRPV1 allosteric modulator as described herein operably linked to a promoter. In some embodiments, a vector comprising a nucleic acid encoding the TRPV1 allosteric modulator is provided. In certain embodiments, the expression vector contains a selectable marker gene to allow the selection of transformed host cells. Selection genes are well known in the art and will vary with the host cell used. Suitable selection genes can include, for example, genes coding for ampicillin and/or tetracycline resistance, which enables cells transformed with these vectors to grow in the presence of these antibiotics.

In some embodiments, a nucleic acid encoding a TRPV1 allosteric modulator is introduced into a cell, either alone or in combination with a vector. By “introduced into,” it is meant that the nucleic acids enter the cells in a manner suitable for subsequent integration, amplification, and/or expression of the nucleic acid. The method of introduction is largely dictated by the targeted cell type. Exemplary methods include CaPO₄ precipitation, liposome fusion, LIPOFECTIN®, electroporation, heat shock, viral infection, and the like.

In some embodiments, prokaryotes are used as host cells for initial cloning steps. Other host cells include, but are not limited to, eukaryotic (e.g., mammalian, plant and insect cells), or prokaryotic (bacterial) cells. Exemplary host cells include, but are not limited to, Escherichia coli, Saccharomyces cerevisiae, Pichia pastoris, Sf9 insect cells, and CHO cells. They are particularly useful for rapid production of large amounts of DNA, for production of single-stranded DNA templates used for site-directed mutagenesis, for screening many constructs simultaneously, and for DNA sequencing of the constructs generated. Suitable prokaryotic host cells include E. coli K12 strain 94 (ATCC No. 31,446), E. coli strain W3110 (ATCC No. 27,325), E. coli K12 strain DG116 (ATCC No. 53,606), E. coli X1776 (ATCC No. 31,537), and E. coli B; and other strains of E. coli, such as HB101, JM101, NM522, NM538, and NM539. Many other species and genera of prokaryotes including bacilli such as Bacillus subtilis, other enterobacteriaceae such as Salmonella typhimurium or Serratia marcesans, and various Pseuaomonoas species can all be used as hosts. Prokaryotic host cells or other host cells with rigid cell walls are typically transformed using the calcium chloride method as described in Green and Sambrook et al., supra. Alternatively, electroporation can be used for transformation of these cells. Prokaryote transformation techniques are set forth in, for example Dower, in Genetic Engineering, Principles and Methods 12:275-296 (Plenum Publishing Corp., 1990); Hanahan et al., Meth. Enzymol., 204:63, 1991. Plasmids typically used for transformation of E. coli include pBR322, pUCI8, pUCI9, pUCI18, pUCI19, and Bluescript M13, all ofwhich are described by Green and Sambrook et al., supra. However, many other suitable vectors are available as well.

Accordingly, some embodiments of the present disclosure provide a host cell comprising a nucleic acid encoding a TRPV1 allosteric modulator, an expression cassette, or a vector as described herein. The host cells can be prokaryotic or eukaryotic. The host cells can be mammalian, plant, bacteria, or insect cells. In some embodiments, a TRPV1 allosteric modulator is produced by culturing a host cell transformed with an expression vector containing a nucleic acid encoding a TRPV1 allosteric modulator, under the appropriate conditions to induce or cause expression of the TRP V1 allosteric modulator. Methods of culturing transformed host cells under conditions suitable for protein expression are well-known in the art (see, e.g., Green and Sambrook et al., supra). Suitable host cells for production of the peptide constructs from lambda pL promoter-containing plasmid vectors include E. coli strain DC 16 (ATCC No. 53606) (see U.S. Pat. No. 5,079,352 and Lawyer, F. C. et al., PCR Methods and Applications 2:275-87, 1993, which are both incorporated herein by reference). Suitable host cells for production of the TRPV1 allosteric modulator from T7 promoter-containing plasmid vectors include E. coli strain BL21 (DE3) and related lysogens (see, e.g., U.S. Pat. No. 5,693,489). Following expression, a TRPV1 allosteric modulator can be harvested and isolated.

Alternatively, TRPV1 allosteric modulators as described herein may be synthesized by solid-phase peptide synthesis methods, during which N-α-protected amino acids having protected side chains are added stepwise to a growing polypeptide chain linked by its C-terminus to a solid support, e.g. polystyrene beads. Various chemistries, resins, protecting groups, protected amino acids and reagents can be employed as described, for example, by Barany and Merrifield, “Solid-Phase Peptide Synthesis,” in The Peptides: Analysis, Synthesis, Biology Gross and Meienhofer (eds.), Academic Press, N.Y., vol. 2, pp. 3-284 (1980); Atherton et al., Solid Phase Peptide Synthesis: A Practical Approach, IRL Press (1989); Bodanszky, Peptide Chemistry, A Practical Textbook, 2nd Ed., Springer-Verlag (1993)); and Chan et al. Fmoc Solid Phase Peptide Synthesis: A Practical Approach, Oxford University Press (2000).

Non-limiting examples of support materials for solid-phase peptide synthesis include polystyrene (e.g., microporous polystyrene resin, mesoporous polystyrene resin, macroporous polystyrene resin; including commercially-available Wang resins, Rink amide resins, and trityl resins), glass, polysaccharides (e.g., cellulose, agarose), polyacrylamide resins, polyethylene glycol, or copolymer resins (e.g., comprising polyethylene glycol, polystyrene, etc.). The solid support may have any suitable form factor. For example, the solid support can be in the form of beads, particles, fibers, or in any other suitable form factor. Non-limiting examples of protecting groups (e.g., N-terminal protecting groups) include Fmoc, Boc, allyloxycarbonyl (Alloc), benzyloxycarbonyl(Z), and photolabile protecting groups. Sidechain protecting groups include, but are not limited to, Fmoc; Boc; cyclohexyloxycarbonyl(Hoc); allyloxycarbonyl (Alloc); mesityl-2-sulfonyl(Mts); 4-(N-methylamino)butanoyl(Nmbu); 2,4-dimethylpent-3-yloxycarbonyl(Doc); 1-(4,4-dimethyl-2,6-dioxocyclohex-l-ylidene)-3-ethyl(Dde); 1-(4,4-dimethyl-2,6-dioxocyclohex-l-ylidene)-3-methylbutyl(ivDde); 4-methyltrityl(Mtt). Additional protecting groups and methods for their addition and removal from supported peptides are described, for example, by Isidro-Llobet et al. Chem. Rev. 2009, 19: 2455-2504.

A base may be used to activate or complete the activation of amino acids prior to exposing the amino acids to immobilized peptides. In some embodiments, the base is a non-nucleophilic bases, such as triisopropylethylamine, N,N-diisopropylethylamine, certain tertiary amines, or collidine, that are non-reactive to or react slowly with protected peptides to remove protecting groups. A coupling agent may be used to form a bond with the C-terminus of an amino acid to facilitate the coupling reaction and the formation of an amide bond. The coupling agent may be used to form activated amino acids prior to exposing the amino acids to immobilized peptides. Any suitable coupling agent may be used. In some embodiments, the coupling agent is a carbodiimide, a guanidinium salt, a phosphonium salt, or a uronium salt. Examples of carbodiimnides include, but are not limited to, N,N-dicyclohexylcarbodiimide (DCC), 1-ethyl-3-(3-dimethylaminopropyl)carbodiimide (EDC) and the like. Examples of phosphonium salts include, but are not limited to, such as benzotriazol-l-yloxy)tripyrrolidinophosphonium hexafluorophosphate (PyBOP); and the like. Examples of guanidinium/uronium salts include, but are not limited to, O-(benzotriazol-l-yl)-N,N,N′,N′-tetramethyluronium hexafluorophosphate (HBTU); 2(7-aza-H-benzotriazole-1-yl)-1,1,3,3-tetramethyluronium hexafluorophosphate (HATU) 1-[(1-(cyano-2-ethoxy-2-oxoethylideneaminooxy)dimethylaminomorpholino)]uronium hexafluorophosphate (COMU); aand the like.

### III. Pharmaceutical Compositions

Some embodiments of the present disclosure provide pharmaceutical compositions containing one or more TRPV1 allosteric modulators as described herein and one or more pharmaceutically acceptable excipients. The pharmaceutical compositions can be prepared by any of the methods well known in the art of pharmacy and drug delivery. In general, methods of preparing the compositions include the step of bringing a TRPV1 allosteric modulator and any other active ingredients into association with a carrier containing one or more accessory ingredients. The pharmaceutical compositions are typically prepared by uniformly and intimately bringing the active ingredient(s) into association with a liquid carrier or a finely divided solid carrier or both, and then, if necessary, shaping the product into the desired formulation. The compositions can be conveniently prepared and/or packaged in unit dosage form. In addition to the TRPV1 allosteric modulator, pharmaceutical compositions provided herein may also contain additional active ingredients such as analgesic agents, NSAIDs, antihistamines, steroids, and anti-cancer agents as described below.

Pharmaceutical compositions containing TRPV1 allosteric modulators can be in the form of aqueous or oleaginous solutions and suspensions (e.g. sterile injectable solutions for intravenous, intraperitoneal, intramuscular, intralesional, subcutaneous, or intrathecal injection; or sterile solutions or suspensions for administration as a nasal spray or nasal drops). Such preparations can be formulated using non-toxic parenterally-acceptable vehicles including water, Ringer's solution, and isotonic sodium chloride solution, and acceptable solvents such as 1,3-butane diol. In addition, sterile, fixed oils can be used as a solvent or suspending medium. For this purpose any bland fixed oil can be employed including synthetic monoglycerides, diglycerides, or triglycerides.

Aqueous suspensions can contain one or more TRPV1 modulators in admixture with excipients including, but not limited to: suspending agents such as sodium carboxymethylcellulose, methylcellulose, oleagino-propylmethylcellulose, sodium alginate, polyvinyl-pyrrolidone, gum tragacanth and gum acacia; dispersing or wetting agents such as lecithin, polyoxyethylene stearate, and polyethylene sorbitan monooleate; and preservatives such as ethyl, n-propyl, and p-hydroxybenzoate. Dispersible powders and granules (suitable for preparation of an aqueous suspension by the addition of water) can contain one or more TRPV1 allosteric modulators in admixture with a dispersing agent, wetting agent, suspending agent, or combinations thereof. Oily suspensions can be formulated by suspending a TRPV1 allosteric modulator in a vegetable oil (e.g., arachis oil, olive oil, sesame oil or coconut oil), or in a mineral oil (e.g., liquid paraffin). Oily suspensions can contain one or more thickening agents, for example beeswax, hard paraffin, or cetyl alcohol. These compositions can be preserved by the addition of an anti-oxidant such as ascorbic acid.

Pharmaceutical compositions containing TRPV1 allosteric modulators can also be formulated for oral use. Suitable compositions for oral administration include, but are not limited to, tablets, troches, lozenges, aqueous or oily suspensions, dispersible powders or granules, emulsions, hard or soft capsules, syrups, elixirs, solutions, buccal patches, oral gels, chewing gums, chewable tablets, effervescent powders, and effervescent tablets. Compositions for oral administration can be formulated according to any method known to those of skill in the art Such compositions can contain one or more agents selected from sweetening agents, flavoring agents, coloring agents, antioxidants, and preserving agents in order to provide pharmaceutically elegant and palatable preparations.

Tablets generally contain the active ingredient in admixture with non-toxic pharmaceutically acceptable excipients, including: inert diluents, such as cellulose, silicon dioxide, aluminum oxide, calcium carbonate, sodium carbonate, glucose, mannitol, sorbitol, lactose, calcium phosphate, and sodium phosphate: granulating and disintegrating agents, such as corn starch and alginic acid; binding agents, such as polyvinylpyrrolidone (PVP), cellulose, polyethylene glycol (PEG), starch, gelatin, and acacia; and lubricating agents such as magnesium stearate, stearic acid, and tale. The tablets can be uncoated or coated, enterically or otherwise, by known techniques to delay disintegration and absorption in the gastrointestinal tract and thereby provide a sustained action over a longer period. For example, a time delay material such as glyceryl monostearate or glyceryl distearate can be employed. Tablets can also be coated with a semi-permeable membrane and optional polymeric osmogents according to known techniques to form osmotic pump compositions for controlled release.

Compositions for oral administration can be formulated as hard gelatin capsules wherein the active ingredient is mixed with an inert solid diluent (such as calcium carbonate, calcium phosphate, or kaolin), or as soft gelatin capsules wherein the active ingredient is mixed with water or an oil medium (such as peanut oil, liquid paraffin, or olive oil).

TRPV1 modulators as described herein can also be administered topically as a solution, ointment, cream, gel, or the like. Still further, transdermal delivery of the TRPV1 modulators can be accomplished by means of iontophoretic patches and the like.

In some embodiments, messenger RNAs encoding the TRPV1 allosteric modulators can be delivered as pharmaceutical compositions containing one or more excipients for increasing stability; increasing cell transfection; permitting the sustained or delayed release (e.g., from a depot formulation); altering the biodistribution (e.g., target to specific tissues or cell types); increasing the translation of encoded TRPV1 modulator in vivo; and/or altering the release profile of encoded TRPV1 modulator in vivo. Such excipients may include solvents, dispersion media, diluents, dispersion or suspension aids, surface active agents, isotonic agents, thickening or emulsifying agents, preservatives. mRNA formulations may also include, without limitation, lipidoids, liposomes, lipid nanoparticles, polymers, lipoplexes, core-shell nanoparticles, peptides, proteins, hyaluronidase, nanoparticle mimics and combinations thereof.

In some embodiments, pharmaceutical compositions for mRNA delivery may include liposomes such as those formed from the synthesis of stabilized plasmid-lipid particles (SPLP) or stabilized nucleic acid lipid particle (SNALP) that have been previously described and shown to be suitable for oligonucleotide delivery in vitro and in vivo (see e.g., U.S. Pat. No. 8,283,333, which is incorporated herein by reference in its entirety). Such liposomes may contain 3, 4, or more lipid components in addition to the mRNA. As an example a liposome can contain, but is not limited to, 50-60% cholesterol, 15-25% disteroylphosphatidyl choline (DSPC), 5-15% PEG-S-DSG, and 10-20% 1,2-dioleyloxy-N,N-dimethylaminopropane (DODMA). Cationic lipids such as 1,2-distearloxy-N,N-dimethylaminopropane (DSDMA), DODMA, DLin-DMA, or 1,2-dilinolenyloxy-3-dimethylaminopropane (DLenDMA), may also be included in the liposomes.

In some embodiments, a polynucleotide encoding a TRPV1 modulator may be formulated in a cationic oil-in-water emulsion where the emulsion particle comprises an oil core and a cationic lipid which can interact with the polynucleotide anchoring the molecule to the emulsion particle. Alternatively, the polynucleotide may be formulated in a water-in-oil emulsion comprising a continuous hydrophobic phase in which the hydrophilic phase is dispersed. (see, e.g., WO2012/006380, which is incorporated by reference in its entirety.

In some embodiments, the compositions include a polynucleotide encoding a TRPV1 modulator and a poloxamer. For example, the polynucleotide may be used in a gene delivery composition with the poloxamer described in U.S. Pub. No. 20100004313, the contents of which are herein incorporated by reference in its entirety. Polymer formulations may be stabilized by contacting the polymer formulation, which may include a cationic carrier, with a cationic lipopolymer which may ne covalently linked to cholesterol and polyethylene glycol groups. The cationic carrier may include, but is not limited to, polyethylenimine, poly(trimethylenimine), poly(tetramethylenimine), polypropylenimine, aminogycoside-polyamine, dideoxy-diamino-b-cyclodextrin, spermine, spermidine, poly(2-dimethylamino)ethyl methacrylate, poly(lysine), poly(histidine), poly(arginine), cationized gelatin, chitosan, cationic lipids as described above, and combinations thereof.

### IV. Methods for TRPV1 Stimulation and Disease Treatment

Also provided herein are methods for stimulating TRPV1 activity in a cell. The methods include contacting the cell with an effective amount of a peptide construct as described above. “Contacting” cells may include addition of a TRPV1 allosteric modulator to a cell culture in vitro, inducing expression of the TRPV1 allosteric modulator in cell culture using a nucleic acid or vector as described above, administering a TRPV1 allosteric modulator to a subject (e.g., in conjunction with a pharmaceutical composition as described above), or inducing expression of the TRPV1 allosteric modulator in the subject. Stimulating TRPV1 generally includes contacting the cells with an amount of the TRPV1 allosteric modulator to increase the level of TRPV1 activity as compared to the level of TRPV1 activity in the absence of the TRPV1 allosteric modulator. For example, contacting the cells with the TRPV1 allosteric modulator can result in activity increases ranging from about 1% to about 99% or higher, e.g., from about 100-200%, or from about 100-250%, or from about 100%-500%. In some embodiments, TRPV1 activity may be increased by an order of magnitude or several orders of magnitude. TRPV1 activity levels may be assessed by a number of techniques, include via calcium imaging or electrophysiological studies as described in more detail below.

Also provided herein are methods for treatment of conditions associated with TRPV1 activity including, but not limited to, pain, itch, cancer, inflammation, atopic dermatitis, chemesthesis, and sensory disorders (e.g., loss of taste and/or smell). The methods include administering an effective amount of a TRPV1 allosteric modulator, or a nucleic acid encoding; the TRPV1 allosteric modulator, to a subject in need thereof. TRPV1 allosteric modulators can be administered at any suitable dose in the methods provided herein. In general, a TRPV1 allosteric modulator is administered at a dose ranging from about 1 microgram to about 1000 milligrams per kilogram of a subject's body weight (i.e., about 0.001-1000 mg/kg). The dose of TRPV1 allosteric modulator can be, for example, about 0.001-1000 mug/kg, or about 0.01-500 mg/kg, or about 0.01-250 mg/kg, or about 0.01-100 mg/kg, or about 0.1-100 mg/kg, or about 0.1-50 mg/kg, or about 0.1-10 mg/kg, or about 0.5-1000 mg/kg, or about 0.5-750 mg/kg, or about 0.750-750 mg/kg. The dose of TRPV1 allosteric modulator can be about 0.001, 0.005, 0.01, 0.05, 0.1, 0.5, 1, 5, 25, 50, 75, 100, 200, 250, 300, 400, 500, 600, 700, 800, 900, or 1000 mg/kg. The dosages can be varied depending upon the requirements of the patient, the severity of the infection being treated, and the particular formulation being administered. The dose administered to a patient should be sufficient to result in a beneficial therapeutic response in the patient. The size of the dose will also be determined by the existence, nature, and extent of any adverse side-effects that accompany the administration of the drug in a particular patient. Determination of the proper dosage for a particular situation is within the skill of the typical practitioner. The total dosage can be divided and administered in portions over a period of time suitable to treat to the disease or condition.

In some embodiments, a TRPV1 allosteric modulator is administered locally via injection to a target organ or tissue (e.g. an arthritic joint; a psoriatic plaque; or a tumor). In some such embodiments, dosages in the range of 1 pig to 50 mg (e.g., 1 μg-10 mg, 10 μg-10 mg, or 100 μg-1 mg) may be employed.

Animal studies, such as mouse studies described in this disclosure, may be useful in determining dosing for humans. For example, an average mouse weighs 0.025 kg. Systemically administering 0.025, 0.05, 0.1 and 0.2 μg of a TRPV1 allosteric modulator according to the present disclosure may therefore correspond to a dose range of 1, 2, 4, and 8 μg of the TRPV1 allosteric modulator/kg. If an average human adult is assumed to have a weight of 70 kg, the corresponding systemic human dosage would be 70, 140, 280, and 560 μg of TRPV1 allosteric modulator. Local dosages may be determined in similar fashion by considering the local mass of a target tissue e.g., a tissue mass of 0.1-0.5 kg surrounding a human joint. Dosages for other active agents may be determined in similar fashion.

TRPV1 allosteric modulators can be administered for periods of time which will vary depending upon the nature of the infection, its severity, and the overall condition of the subject to whom the TRPV1 allosteric modulator is administered. Administration can be conducted, for example, hourly, every 2 hours, three hours, four hours, six hours, eight hours, or twice daily including every 12 hours, or any intervening interval thereof. Administration can be conducted once daily, or once every 36 hours or 48 hours, or once every month or several months. Following treatment, a subject can be monitored for changes in his or her condition and for alleviation of the symptoms of the disorder. The dosage of the TRPV1 allosteric modulator can either be increased in the event the subject does not respond significantly to a particular dosage level, or the dose can be decreased if an alleviation of the symptoms of the disorder is observed, or if the disorder has been remedied, or if unacceptable side effects are seen with a particular dosage. Administration of the TRPV1 allosteric modulator may be conducted over periods of time ranging from a few days to several weeks, months, or years.

In some embodiments, the methods may be used for the treatment of pain. The terms “neuropathic pain” or “neurogenic pain” can be used interchangeably and refer to pain that arises from direct stimulation of nervous tissue itself, central or peripheral and can persist in the absence of stimulus. The sensations that characterize neuropathic pain vary and are often multiple and include burning, gnawing, aching, and shooting. (See, e.g., Rooper and Brown, (2005) Adams and Victor's Principles of Neurology, 8^{th} NY, McGraw-Hill). These damaged nerve fibers send incorrect signals to other pain centers. The impact of nerve fiber injury includes a change in nerve function both at the site of injury and areas around the injury, as well as in the central nervous system. Chronic neuropathic pain often seems to have no obvious cause, however, some common causes may include, but are not limited to, alcoholism, amputation, back, leg and hip problems, chemotherapy, diabetes, facial nerve problems (e.g., trigeminal neuralgia), HIV infection or AIDS, multiple sclerosis, shingles, spine surgery, spinal cord injury, traumatic brain injury, and stroke. For example, one example of neuropathic pain is phantom limb syndrome, which occurs when an arm or leg has been removed because of illness or injury, but the brain still gets pain messages from the nerves that originally carried impulses from the missing limb. In some embodiments, the pain may be inflammatory pain, cancer pain, muscle pain, orthodontic pain, shingles-induced pain, or headache (e.g., migraine). Mechanical allodynia or tactile allodynia, pain induced by normally innocuous mechanical stimulation, is a common feature of chronic pain. Chronic pain due to arthritis and cancer are serious problems in pets and other companion animals in addition to humans. Advantageously, as discussed below, TRPV1 modulators can be used for treatment of pain without causing hyperthermia in subjects to whom the modulators are administered. In some embodiments, for example, the body temperature of the subject may increase by no more than 1° C. above basal body temperature (e.g., less than 0.8° C. or less than 0.5° C. above basal body temperature) upon administration of a TRPV1 modulator to the subject.

In some embodiments, the methods may be used for the treatment of pruritus, commonly referred to as itching. Itch is a major medical problem, and its impact on quality of life can equal that of pain. It represents an unmet medical need as few effective therapies are available. Itch can originate in the peripheral nervous system (dermal or neuropathic) or in the central nervous system (neuropathic, neurogenic, or psychogenic) Itch originating in the skin is considered proprioceptive and can be induced by a variety of stimuli, including mechanical, chemical, thermal, and electrical stimulation. Neuropathic itch can originate at any point along the afferent pathway as a result of damage of the nervous system. They could include diseases or disorders in the central nervous system or peripheral nervous system. Examples of neuropathic itch in origin are nostalgia paresthetica, brachioradial pruritus, brain tumors, multiple sclerosis, peripheral neuropathy, and nerve irritation. Neurogenic itch, which is itch induced centrally but with no neural damage, is often associated with increased accumulation of endogenous opioids and possibly synthetic opioids. Itch is also associated with some psychiatric disorders such as delusions of parasitosis or related obsessive-compulsive disorders, for example neurotic scratching.

TRPV1 allosteric modulators may also be administered in conjunction with other agents useful to treat pain and/or itch, including but not limited to steroids (e.g., cortisone, hydrocortisone) and non-steroidal anti-inflammatory agents (NSAIDs). Examples of NSAIDs include, but are not limited to, aceclofenac, 5-amino salicylic acid, aspirin, celecoxib, dexibuprofen, diclofenac, diflusinal, etodolac, fenoprofen, flufenamic acid, flurbiprofen, ibuprofen, indomethacin, ketoprofen, ketorolac, loxoprofen, mefenamic acid, nabumetone, naproxen, nimesulide, sulindac, and pharmaceutically acceptable salts thereof. Additional analgesic agents such as paracetamol (acetaminophen) and opioid analgesic agents (e.g., fentanyl, sufentanil, carfentanyl, lofentanil, alfentanil, hydromorphone, oxycodone, morphine, hydroxycodone, propoxyphene, pentazocine, methadone, thyridine, butorphanol, buprenorphine, levorphanol, codeine, oxymorphone, meperidine, and the like) may also be administered in conjunction with the TRPV1 modulator. Other active agents which are known to inhibit neuronal excitation may also be administered including, but not limited to, sodium channel blockers, local anaesthetics, modulators of CB2 receptors, potassium channel openers, calcium channel blockers, NMDA-receptor antagonists, GABA receptor modulators, alpha2 adrenoceptor modulators, examples of which are described in WO 2009/000038, which is incorporated herein by reference in its entirety. In some embodiments, treatment of pain may further include administration of a non-steroidal anti-inflammatory agent, an opioid analgesic, acetaminophen, or a combination thereof to the subject.

In some embodiments, treatment of pruritus may further include one or more antihistamines (e.g., diphenhydramine, clemastine, loratadine, hydroxyzine, promethazine, alimemazine, pheniramine, or the like), one or more steroids (e.g. clobetasol propionate, desonide, desoximetasone, fluocinonide, halobetasol propionate, hydrocortisone, triamcinolone acetomide, and the like), one or more anesthetics (e.g., lignocaine, bupivacaine, ropivacaine, procaine, tetracaine, and the like) or a combination thereof to the subject

TRPV1 is known to be expressed in several different neoplastic tissues. In some embodiments, the methods may be used for the treatment of cancers including, but not limited to, chronic myeloid leukemia, hairy cell leukemia, melanoma, multiple myeloma, non-Hodgkin lymphoma, acute myeloid leukemia, prostate cancer (including castration-resistant prostate cancer), chronic lymphocytic leukemia, cutaneous lymphomas, polycythemia vera, relapsed follicular lymphoma, systemic mastocytosis, and testicular cancer (including testicular teratoma). Other cancers include fibrosarcoma myxosarcoma, liposarcoma, chondrosarcoma, osteogenic sarcoma, chordoma, angiosarcoma, endotheliosarcoma, lymphangiosarcoma, lymphangioendothelio-sarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, small cell lung carcinoma, bladder carcinoma, lung cancer, epithelial carcinoma, glioma, glioblastoma multiform, astrocytoma, medulloblastoma, craniopharyngioma, ependymoma, pinealoma, hemangioblastoma, acoustic neuroma, oligodendroglioma, meningioma, skin cancer, neuroblastoma, or retinoblastoma.

In some embodiments, the cancer is a breast cancer, squamous cell carcinoma (e.g., of the tongue), hepatocellular carcinoma, or carcinoma of the bladder (e.g., a transitional carcinoma of the human bladder).

Methods for treating cancer may include administration of one or more anti-cancer agents including, but are not limited to, a chemotherapeutic agent (e.g., carboplatin, paclitaxel, pemetrexed, or the like), a tyrosine kinase inhibitor (e.g., erlotinib, crizotinib, osimertinib, or the like), and an immunotherapeutic agent (e.g., pembrolizumab, nivolumab, durvalumab, atezolizumab, or the like). One or more anti-cancer agents may be administered to a subject prior to administration of a TRPV1 allosteric modulator, concomitantly with administration of a TRPV1 allosteric modulator, or after administration of a TRPV1 allosteric modulator. Ami-cancer agents may be co-formulated with TRPV1 allosteric modulators in pharmaceutical compositions such as those as described above. In some embodiments, methods for treating cancer includes the administration of radiotherapy, e.g., external beam radiation; intensity modulated radiation therapy (IMRT); brachytherapy (internal or implant radiation therapy); stereotactic body radiation therapy (SBRT)/stereotactic ablative radiotherapy (SABR); stereotactic radiosurgery (SRS); or a combination of such techniques. Dosing and administration of the TRPV1 allosteric modulator may be varied as set forth above.

### V. Examples

### Example 1. Computational Design of Protein Binders to the ARD

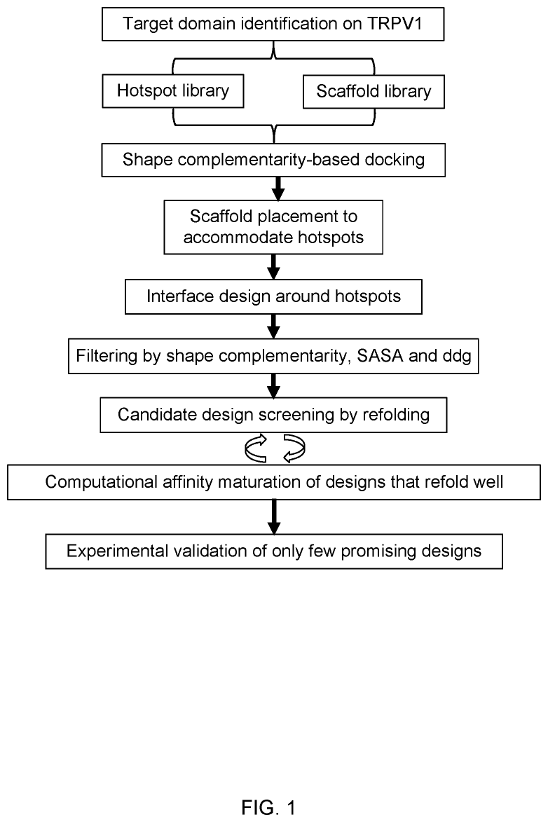

Protein binders to the ARD were de novo designed following the hotspot centric stratagem described previously (FIG. 1) (17). Briefly, the structure of TRPV1 ARD (PDB ID: 2PNN) was first cleaned and relaxed in the Rosetta suite version 3.4 (16) See Script 1 above. Then each of the natural amino acids was docked to ARD in Rosetta (31). Two Phe residues bound well to the ARD so that they were chosen as the hotspots for subsequent protein design. The inverse rotamer library of these two residues was generated using RosettaScripts (24) (Script 2 above). The scaffold library was generated by selecting protein structures from the PDB database with the following criteria:
1. There is not any DNA, RNA or disulfide bond.
2. There is only one protein chain and stoichiometry is monomer.
3. There are less than 100 residues.
4. It can be expressed in E. Coli.
5. The structure is determined by X-ray at a resolution higher than 2.5 Å.
6. There is no ligand presented in structure.
7. Homologue removal is set at 70% identity.

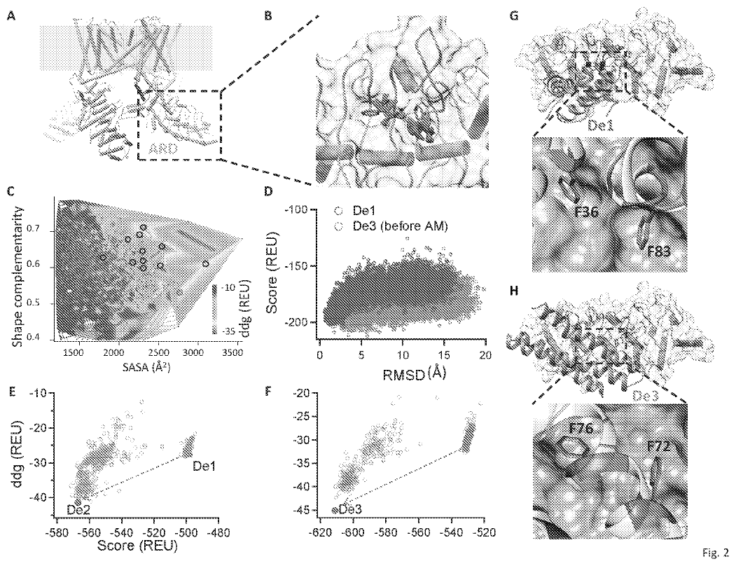

A total of 167 unique protein structures was selected. They were further cleaned and prepacked in Rosetta (Scripts 3 and 4 above). The ARD was then docked to the scaffold library in a coarse-grained manner with the PatchDock software (23) based on protein structure shape complementarity. The patchdocked scaffolds were fused with the two hotspots and then the protein-protein interface was redesigned by RosettaScripts (Script 5 above). These initial designs were assessed by plotting as shown in FIG. 2C. Only the designs with shape complementarity, ddg and SASA larger than 0.6, −25 Rosetta Energy Unit (REU) and 1000 Å², respectively, were kept. 14 designs with unique protein scaffolds were selected. These designs were further ab initio refolded based on their primary sequence in Rosetta. Only two designs exhibited funnel-shaped energy-RMSD distribution, indicating reasonable refolding. There two designs were then subjected to in silico affinity maturation by RosettaScripts (Script 6 above). The designs with the largest ddg and score were selected as the final designs.

All the molecular graphics of protein structure models were rendered by UCSF Chimera (32) software version 1.12 (33).

To positively modulate TRPV1 channel in a precise and domain-specific manner, we chose to design binders to the ARD (FIG. 2A). The major steps in our protein design method are illustrated in FIG. 1, where we employed three steps in computation (colored in blue) to eliminate pseudo positive candidates and increase the success rate of binder design. Similar to the original hotspot centric method (17), to first anchor hotspot residues on the ARD for further design, we docked each natural amino acid to the concave surface of ARD (FIG. 2B). Among these residues, we observed that two phenylalanine residues bound to ARD with favorable binding energy and structural convergence in the sidechain conformation (FIG. 2B), so we employed these two phenylalanine residues as the hotspots. We then selected a pool of protein structures as the candidate scaffolds based on multiple criteria. With Patchdock software (23) and the Rosetta suite (16), we fused the candidate scaffolds with the two hotspots based on protein shape complementarity, and then redesigned the residues on candidate scaffolds forming the interface with TRPV1 ARD for larger binding energy (the score term ddg in Rosetta suite). After this step, thousands of initial candidate binder designs were generated (FIG. 2C, dots in grey). To increase the success rate of our design trials in the downstream experimental validations, we needed to eliminate pseudo positive designs while keep the real promising designs in these initial candidates.

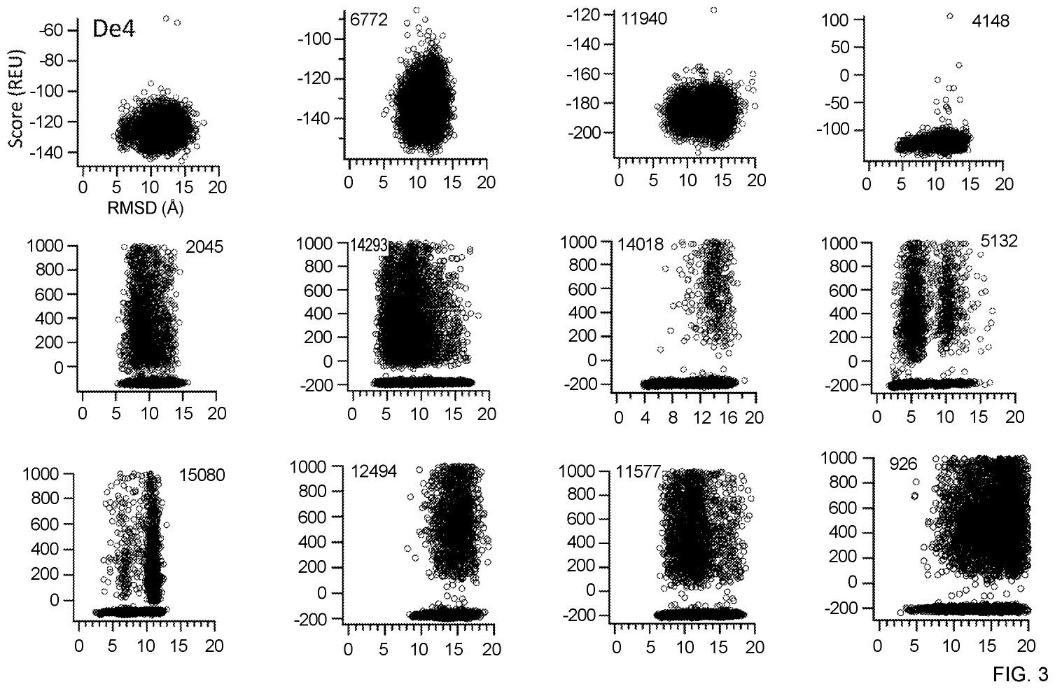

Toward this goal, we improved the original hotspot centric method in three consecutive steps. First, we evaluated the candidate designs multi-metrically (FIG. 2C. The candidate designs were sorted based on their shape complementarity, binding energy (ddg) and solvent accessible surface area (SASA) of the interface (FIG. 2C). Only the candidates with shape complementarity ddg and SASA larger than 0.6, −25 Rosetta Energy Unit (REU) and 1000 Å², respectively, were kept. Among these candidates, designs with top 50 ddg were further selected. We inspected these candidates and found 14 unique scaffolds. In the second step, we evaluated whether these 14 candidates can refold towards the designed structures. Because when the scaffolds were fused with the hotspots and their interface residues were redesigned, the protein sequence homology of a candidate was reduced to about 70% of its scaffold, rising the risk of large changes in its structure. We performed the classic ab initio modeling of these candidates based on their designed primary sequence only. We observed that only two (De1 and precursor of De3) out of the 14 candidates could refold to the designed three-dimensional structures with a funnel-shaped energy distribution (FIG. 21)), while the other 12 designs exhibited either positive score which indicated no proper refolding, or non-funnel-shaped score distribution in refolding (FIG. 3). In the third step, we performed in silico affinity maturation in RosettaScripts (24) to simultaneously optimize the binding energy (ddg) and the stability (total score) of these two designs. For instance, De2 was the optimized version of De1, where both its ddg and score were better than De1. The precursor of De3 was optimized to get De3 (FIGS. 2E and 2F). In De1 and De3, the hotspot residues (F36 and F83 of De1, F72 and F76 of De3, respectively) were designed to interact with the concave surface of the ARD through hydrophobic interactions (FIGS. 2G and 2H). Finally, we chose three designs (De1, De2, De3) for experimental validation (Table 4), and another design (De4) as a negative control because this design cannot refold well (FIG. 3).

TABLE 4

| | | Number | Number | Total | | Total | | |
|---|---|---|---|---|---|---|---|---|
| Design | Scaffold | of | of | Score | ddg | SASA | Hydrophobic | Shape |
| Name | PDB | Residues | Mutations | (REU) | (REU) | (Å) | area (Å) | complementarity |
| De1 | IQZM | 93 | 24 | −497.304 | −31.13 | 1930.48 | 1367.27 | 0.6743 |
| De2 | IQZM | 93 | 26 | −567.079 | −40.64 | 2253.36 | 1689.67 | 0.6853 |
| De3 | 3ONJ | 96 | 26 | −608.476 | −45.18 | 2991.22 | 2049.31 | 0.6355 |
| De4 | 2HDZ | 65 | 22 | −408.786 | −37.35 | 2311.28 | 1843.63 | 0.7126 |

### Example 2. Fluorescence Imaging and FRET Quantification Demonstrate that Designed Proteins Bind to TRPV1

To experimentally test whether our designed proteins work as PAM of TRPV1, we first tested whether they could interact with TRPV1 channel in live cells. We genetically fused YFP and CFP to the C termini of TRPV1 and our designs, respectively.

Super-resolution fluorescence microscopy was performed with the Zeiss LSM 880 fluorescence microscope with Airyscan. YFP fused to TRPV1 and CFP fused to the designed proteins were excited by laser line at 488 nm and 458 nm, respectively. We used a 60× oil-immersion objective (NA 1.42) in these experiments. Fluorescence imaging was performed with the default settings for CFP and YFP channels in the microscope controlled by the Zeiss ZEN software. Overlay of YFP- and CFP-channel images was also done in ZEN.

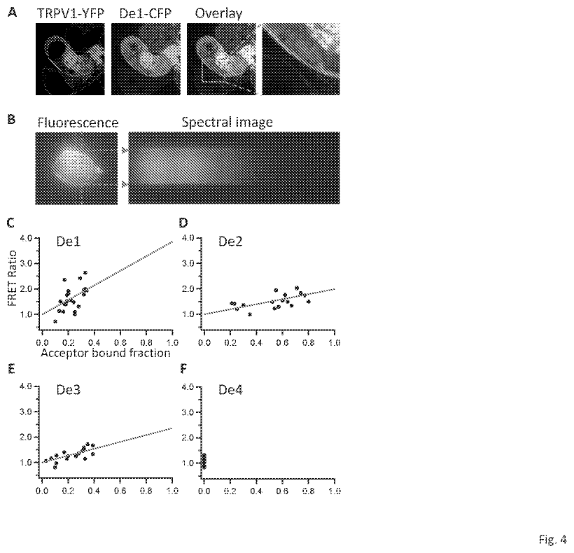

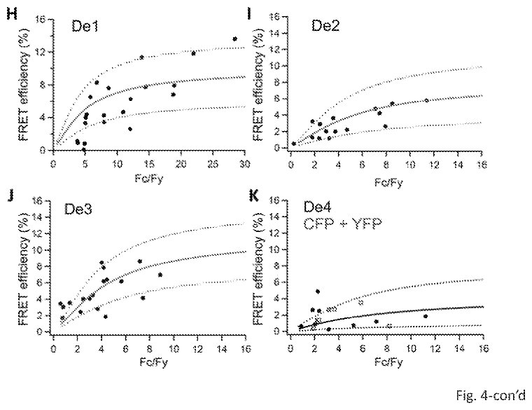

For spectroscopic imaging in FRET experiments, the imaging system was built upon a Nikon TE2000-U microscope. The excitation light was generated by an Ar laser. The duration of light exposure was controlled by a computer-driven mechanical shutter (Uniblitz). A spectrograph (Acton SpectraPro 2150i) was used in conjunction with a CCD camera (Roper Cascade 128B). In this recording mode two filter cubes (Chroma) were used to collect spectroscopic images from each cell (excitation, dichroic): cube 1. D436/20, 455dclp; cube II, HQ500/20, Q5151p. No emission filter was used in these cubes. Under our experimental conditions, auto fluorescence from untransfected cells was negligible. Fluorescence imaging and analysis were done using the MetaMorph software (Universal Imaging). User-designed macros were used for automatic collection of the bright field cell image, the fluorescence cell image, and the spectroscopic image. Emission spectra were collected from the plasma membrane of the cell by positioning the spectrograph slit across a cell and recording the fluorescence intensity at the position corresponding to the membrane region (FIG. 4B, dotted lines in red); the same slit position applied to both the spectrum taken with the CFP excitation and the spectrum taken with the YFP excitation. Using this approach, the spectral and positional information are well preserved, thus allowing reliable quantification of FRET efficiency specifically from the cell membrane. Spectra were corrected for background light, which was estimated from the blank region of the same image. FRET efficiency, as reflected by the FRET ratio, was calculated from the increase in YFP emission due to energy transfer as described in our previous study (34). CFP emission was separated from YFP emission by fitting of standard spectra acquired from cells expressing YFP or CFP only.

When De1 and TRPV1 channel were co-transfected and expressed in HEK293 cells, we observed clear co-localization of these molecules on cell membrane with the Airyscan super resolution imaging (FIG. 4A), indicating our designs were in close proximity to TRPV1. Due to the limitation in spatial resolution of the Airyscan imaging (about 120 nm laterally) (25), we further performed FRET imaging because FRET occurs only when the distance between donor and acceptor fluorophores is less than 10 nano meters (26). By imaging the emission spectra of design-CFP and TRPV1-YFP co-expressed in cells, we detected positive FRET signals from all three designs (De1, De2 and De3), while no FRET occurred between De4, the negative control, and TRPV1 (FIG. 4C and Table 5).

TABLE 5

| Design | Apparent K_{D} | FRET ratio max |
|---|---|---|
| De1 | 1.2*10⁵ | 3.86 |
| De2 | 7.5*10⁴ | 2.35 |
| De3 | 1.1*10⁴ | 1.99 |
| De4 | 9.5*10⁹ | n.a. |

We further quantified the FRET efficiency between the designed protein and TRPV1 using the spectraFRET method we previously used to study TRP channels.[30] We observed that De1, De2 and De3 show FRET efficiency values of 9-2%, 7.4%, and 10.9%, respectively (FIG. 4G-I), much larger than that of De4 (3.8%, FIG. 4J), which was indistinguishable from the negative control where standalone CFP and YFP proteins were coexpressed (FIG. 4J, closed circles for De4 and open squares for the CFP+YFP negative control). Therefore, our imaging experiments showed that the designs interact with TRPV1 channel in live cells.

### Example 3. Surface Plasmon Resonance Measurements Demonstrates that Designed Proteins Bind to TRVP1 ARD

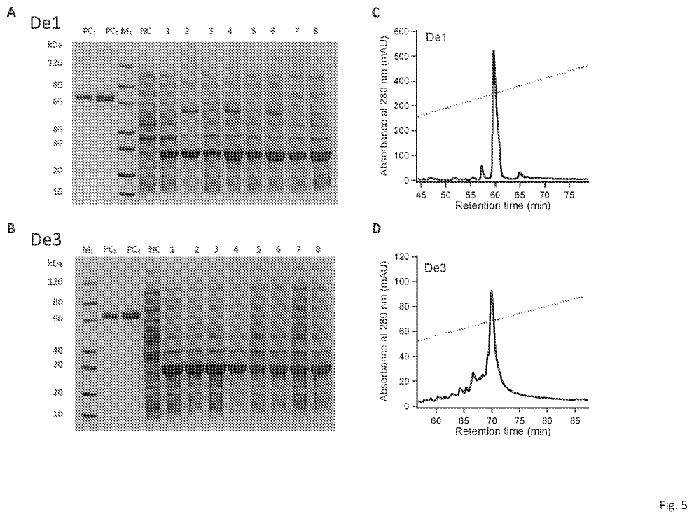

To further quantify the binding affinity of the designs to the ARD, we expressed and purified both designed proteins and the ARD in E. coli. The DNA sequence of each designed protein and the ARD of TRPV1 (see their primary protein sequences in Table 6) was synthesized and inserted into the pET-32a plasmid after codon optimization for protein expression in bacteria E. coli BL21 (DE3) was transformed with the recombinant plasmids. A single colony was inoculated into media containing ampicillin; cultures were incubated in 37° C. at 200 rpm. Once cell density reached to OD 0.8-1.0 at 600 inn, IPTG was introduced for induction. SDS-PAGE was used to monitor the expression. A range of expression conditions were tested as shown in FIG. 5, where the best protein expression condition was determined to be induction with 0.5 mM IPTG and expression at 15° C. for four hours in LB medium. The protein expression was stopped by centrifugation at 8000 rpm for 30 min. We collected cells and resuspended in resuspension solution containing 50 mM Tris-HCl, 150 mM NaCl, 10% glycerol and protease inhibitor (pH 7.4). The cells were lysed by sonication. The supernatant after centrifugation was kept for future purifications. Taget proteins were obtained by Ni column, and then further purified by the reversed phase HPLC on a Jupiter C4 column (10×250 mm, Phenomenex, Torrance, CA, USA). The system was equilibrated by 0.1% TFA (Solution A). The proteins were separated with gradient of 0.1% TFA acetonitrile (Solution B) at a rate of 1.5 mL/min. The proteins of ARD, De1 and De3 were robustly and abundantly produced in E. coli. (FIG. 5), while the expression of De2 was minimal in bacterial cells.

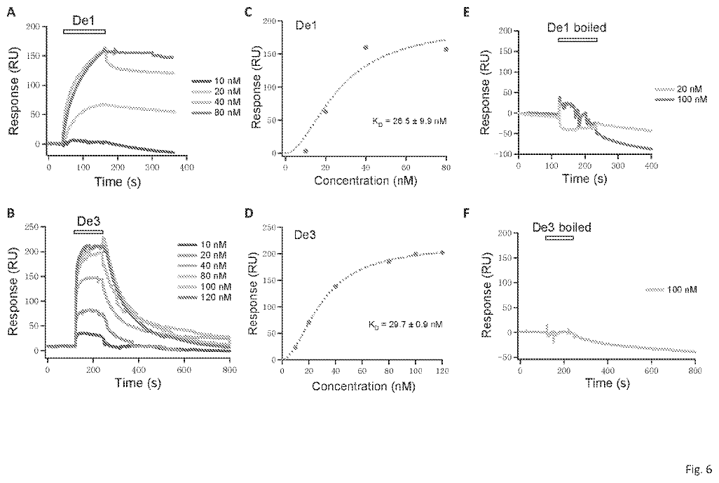

We then quantified the binding of De1 and De3 to the ARD protein fixed on the sensor chip by surface plasma resonance (SPR) (FIGS. 6A and 6B). The purified ARD was immobilized on the CM5 SPR sensor chip by an amine-coupling procedure. SPR experiments were conducted on the Biacore T3000 instrument (GE healthcare). The designed proteins were perfused as analytes. Steady-state SPR responses were measured and used to determine the K_{D} values. In all experiments, the analysis was performed at 25° C., with an association time of 120 see at a flow rate of 20 μL/min.

We observed that De and De3 bound with the ARD in a concentration-dependent manner, yielding K_{D} values of 26.5±9.9 nM and 29.7±0.9 nM, respectively (FIGS. 6C and 6D). As negative controls, when De1 and De3 proteins were boiled to disrupt their three-dimensional structures, the binding with the ARD was completely abolished (FIGS. 6E and 6F). These results demonstrated that De1 and De3 can potently bind with the target ARD as we designed.

TABLE 6

| Protein | Primary Sequence |
|---|---|
| Del | NGYSSEDKLRIAKRHLLPKQIERNALKKGELFVSEFAIL |
| (SEQ ID | GIILFYTDEAGVRGLEREISKLCRKAVKQLLLDKSTTSS |
| NO: 8) | HASGFHLHDYLGVMR |
| De2 | NGYWSEDKERIAKRHLLPKQIERNALKKGELKVWEFAIF |
| (SEQ ID | GIILFYTWEAGVRGLEREISKLCRKAVKQLLLDKSTTRS |
| NO: 37) | WASGFHLHDYLGVMR |
| De3 | SLLDSYKLDFWKTSTNAENSLHEAPSQPLSQRNTTLKHV |
| (SEQ ID | EQQQDELFDLLDQMDVEVNNSIGRDADRRWYLFELWFWK |
| NO: 9) | ISIEEEIKRPLQSLVDSG |
| De4 | LPESPRFAQKIWQQSVIGDYLARFKNDRVKALKAMEDSS |
| (SEQ ID | NNSSMHRFAEFFAKAFEDLHRYLEEL |
| NO: 38) | |
| ARD | MSVSAGEKPPRLYDRRSIFDAVAQSNCQELESLLPFLQR |
| (SEQ ID | SKKRLTDSEFKDPETGKTCLLKAMLNLHNGQNDTIALLL |
| NO: 39) | DVARKTDSLKQFVNASYTDSYYKGQTALHIAIERRNMTL |
| | VTLLVENGADVQAAANGDFFKKTKGRPGFYFGELPLSLA |
| | ACTNQLAIVKFLLQNSWQPADISARDSVGNTVLHALVEV |
| | ADNTVDNTKFVTSMYNEILILGAKLHPTLKLEEITNRKG |
| | LTPLALAASSGKIGVLAYILQREIHEPECRHAAAHHHHH |
| | H |

### Example 4. Calcium Imaging Demonstrates that Designed Proteins are Positive Allosteric Modulators of TRPV1

We then investigated whether, upon binding of the designed proteins, TRPV1 activation was positively modulated. Transiently transfected HEK293 cells seeded on 25 mm coverslips were washed twice with an extracellular solution (ECS) containing 140 mM NaCl, 5 mM KCl, 1 mM MgCl₂, 1.8 mM CaCl₂, 10 mM glucose, and 15 mM HEPES (pH 7.4), followed by incubation in 2 ml of ECS supplemented with 2 μM Fluo-4/AM (Kd for Ca²⁺ at 345 nM) and 0.05% Pluronic F-127 (both from Molecular Probes) at room temperature for 60 min. Probenecid (2 mM) was included in all solutions to prevent Fluo-4 leakage from cells. At the end of incubation, cells were washed three times with ECS and incubated in the same solution for another 20 min at room temperature to complete the intracellular hydrolysis process of the AM ester, which converts the non-fluorescent Fluo-4/AM into the fluorescent version Fluo-4.

Coverslip with dye-loaded cells was placed in the quick-release magnetic chamber (Warner) and mounted on the stage of a Nikon Eclipse TE2000-U microscope system equipped with a Roper Cascade 128B CCD camera. Fluo-4 was excited by an Argon laser with a filter set of Z488/10 (excitation), z488rdc (dichroic) and recorded through an emission filter HQ5001p (all from Chroma). The duration of light exposure was controlled by a computer-driven mechanical shutter (Uniblitz). Cell images were acquired sequentially with an exposure period of 200 ms at an interval of 1 s. The shutter and the camera were controlled and synchronized by MetaMorph software (Universal Imaging). Cells pretreated with 1 μM thapsigargin during the dye-loading step (aiming to deplete ER Ca²⁺ store) did not exhibit noticeable difference in fluorescence intensity or kinetics changes compared to untreated cells.

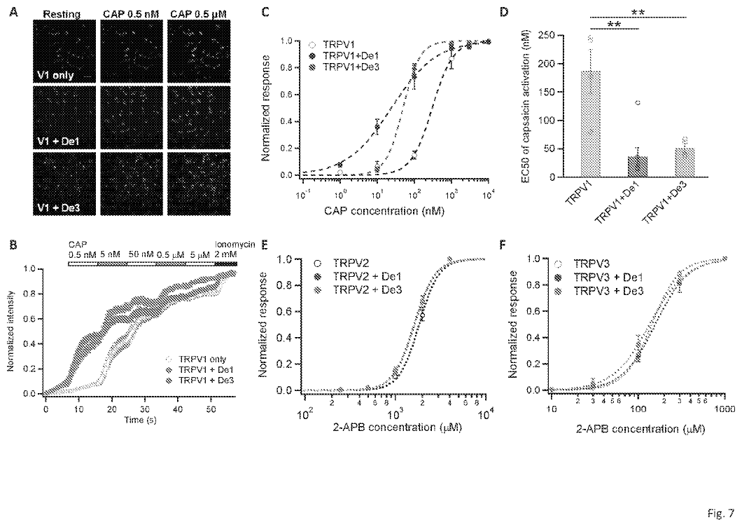

With calcium imaging, we first observed that while 0.5 nM capsaicin did not illicit calcium influx in cells expressing only TRPV1, this low concentration was sufficient to activate TRPV1 channels co-expressed with either De1, or De3 (FIGS. 7A and 7B), indicating the designed proteins enhanced capsaicin sensitivity in TRPV1.

### Example 5. Electrophysiological Study of TRPV1 Positive Allosteric Modulation by Designed Proteins

Patch-clamp recordings were performed with a HEKA EPC10 amplifier controlled by PatchMaster software (HEKA). Whole-cell recordings at ±80 mV were used to test whether an ANAP-incorporated channel was functional. Patch pipettes were prepared from borosilicate glass and fire-polished to resistance of ˜4 MΩ. For whole-cell recording, serial resistance was compensated by 60%. A solution with 130 nM NaCl, 10 mM glucose, 0.2 mM EDTA and 3 mM HEPES (pH 7.2) was used in both bath and pipette for concentration response curve measurements in TRPV1, TRPV2 and TRPV3. To measure calcium-dependent ligand-induced desensitization and tachyphylaxis, 2 mM CaCl₂ was added to the solution. Membrane potential was clamped at ±80 mV. Current was sampled at 10 kHz and filtered at 2.9 kHz. All recordings were performed at room temperature (22° C.) with the maximum variation of 1° C. The capsaicin or 2-APB concentration-dependent activation curves were fit to a Hill equation to obtain the EC50 value and the slope factor.

Ligands were perfused to membrane patch by a gravity-driven system (RSC-200, Bio-Logic). Bath and ligand solution were delivered through separate tubes to minimize the mixing of solutions. Patch pipette was placed in front of the perfusion tube outlet.

Consistent with calcium imaging, patch-clamp recordings revealed that, when either De1 or De3 was co-expressed with TRPV1, the concentration-response curve of capsaicin activation in the absence of extracellular calcium was left-shifted (FIG. 7C), with the EC50 values reduced from 186.1±38.8 nM to 50.9±7.8 nM or 36.1±16.1 nM, respectively (FIG. 7D). In contrast, when the designed proteins were co-expressed with the closely related ARD-containing TRPV2 or TRPV3 channels, no change in ligand activation was observed (FIGS. 7E and 4F), indicating our designs were highly selective for the TRPV1 channel. Therefore, these results indicated our designs can positively and selectively modulate TRPV1 activities.

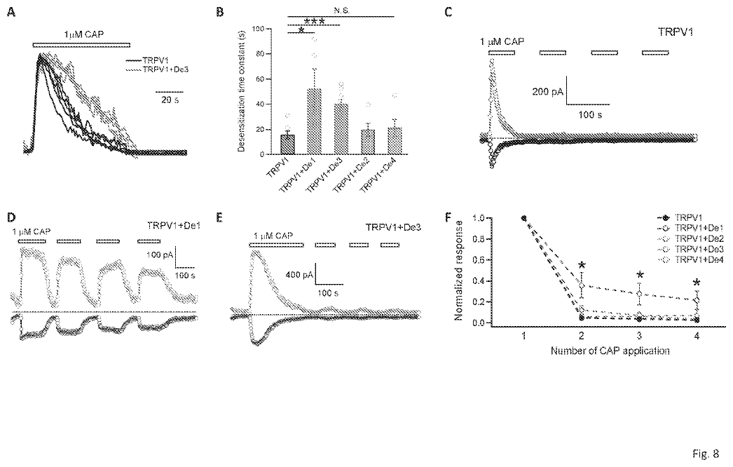

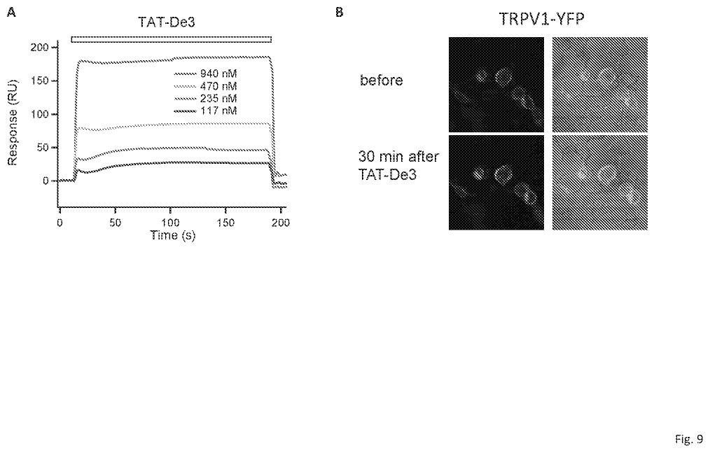

Moreover, ligand activation of TRPV1 was accompanied with an acute desensitization process in the presence of extracellular calcium ions (FIG. 8A, current traces in black), which serves as a negative feedback mechanism to prevent excessive calcium entry through the channel causing calcium overload and cell damages. For our designs to exert analgesic effects, they need to counter such a desensitization during ligand activation. We observed that when either De1 or De3 was co-expressed with TRPV1, current desensitization upon capsaicin activation was significantly slowed down (FIGS. 8A and SB), while no such an effect was observed when De2 or De4 was co-expressed. Besides the acute desensitization in TRPV1 ligand activation, repeated agonist application also led to tachyphylaxis in TRPV1 channel, where the current responses exhibited diminishing amplitudes (13) (FIG. 8C). We observed that in the presence of De1, capsaicin induced tachyphylaxis was reduced (FIGS. 8D and 8F), which again increased calcium entry through the channel. De3 did not reduce tachyphylaxis of TRPV1 (FIGS. 8E and SF). Therefore, both calcium imaging and patch-clamp recording demonstrated that our De1 and De3 are PAMs of TRPV1. Furthermore, the slowed acute desensitization and reduced tachyphylaxis with our designed PAMs indicated more calcium entry through TRPV1 channels, which laid the basis for local calcium overload and analgesia.

### Example 6. Study of Analgesic Effects Exerted by Designed PAMs in Animals

To test whether our designed PAMs have analgesic effects, we performed animal behavior experiments.

Animals. Male Sprague-Dawley rats (200-250 g, Charles River Laboratories, Inc.) were housed under a 12 h light-dark cycle and allowed access to food and water ad libitum. The ambient temperature of the holding and testing; rooms was ˜22° C. All procedures involving animals were carried out in strict compliance with the National Institutes of Health and institutional guidelines for the humane care of animals and were approved by the Animal Care and Use Committee of Zhejiang University (Approval ID: ZJU20190100). All efforts were made to minimize both animal numbers and distress within the experiments.

Animal behavioral measurements. A 10 mM stock solution of MRS1477 was prepared in 100% DMSO and further diluted in vehicle to 2 μg/100 μl (50 μM). Capsaicin was prepared as a 100 mM stock solution in DMSO, stored at −80° C., and was diluted directly into vehicle on the day of the experiments to 4.6 μg/100 μl (150 μM). Capsaicin-only injectates contained an equal amount of DMSO as those with MRS1477. All intraplantar injections were made using a. 29 G×½″, 3/10 cc insulin syringe. The experimenter was blinded to the identity of the injectates in the various behavioral experiments.

Thermal hyperalgesia measurement was performed as reported previously (11, 35). Briefly, unrestrained rats were placed on a clear glass platform which a light beam was applied onto the plantar hind paws of the animals under a small plastic cage which allowed them to move freely. The thermal nociceptive response was defined as the latency between light stimulus onset and paw withdrawal using a feedback-controlled shutdown unit. The intensity of the light stimulus was set such that naive rats responded with a latency of approximately 5 s. Each paw was tested one time. In the absence of a response within a predetermined maximum latency (30 s), the test was terminated to prevent tissue damage. On the day of testing, rats were allowed to habituate for at least 30 min prior to thermal stimulation. Rats were tested prior to intradermal injection to establish a baseline, then at 2, 24, 48, 72 hours, 7, 16 and 21 days post-injection.

Statistics. All experiments have been independently repeated for at least three times. All statistical data are given as mean±SEM. Two-sided Student's t-test was applied to examine the statistical significance. N.S. indicates no significance. *, ** and ***,p<0.05,p<0.01 andp<0.001, respectively.

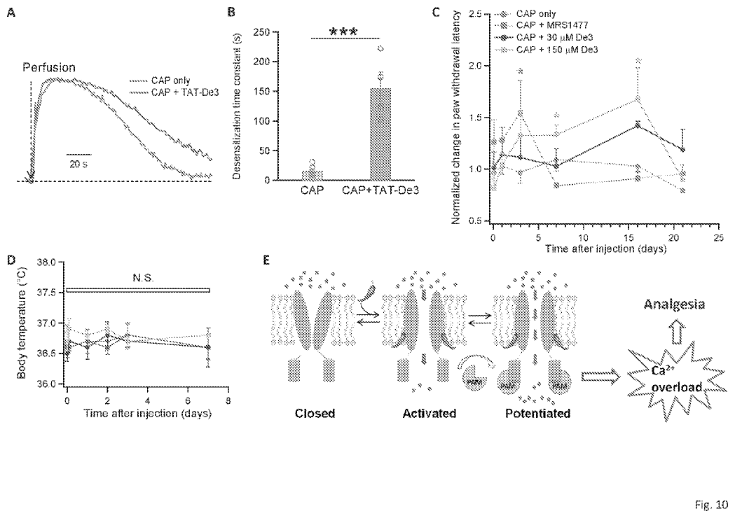

Results. Since the ARD of TRPV1 is located intracellularly, we genetically fused the TAT peptide (RKKRRQRRR) (27) to the N terminus of our De1 and De3 to facilitate their transmembrane delivery. Only TAT-De3 was able to be expressed and purified in E. coli. We observed that the TAT-De3 can still bind to the ARD in SPR experiments, though its affinity was reduced (FIG. 9A). Up to 300 μM TAT-De3 did not disrupt the cell membrane even after 30 min incubation (FIG. 9B). More importantly, TAT-De3 applied extracellularly could diffuse across the cell membrane to slow down capsaicin induced acute current desensitization (FIGS. 10A and 10B) just like when De3 was applied intracellularly (FIGS. 8A and 8B).

We observed significant analgesic effects of TAT-De3 intradermally injected into the hind paw of rats against thermal pain. We used the small molecule PAM MRS1477 as the positive control, because the analgesic effects of this molecule in thermal pain has been demonstrated (11). We used a halogen lamp to heat the hind paw of a rat at a spot less than 20 mm² to above 45° C. to elicit thermal pain. We recorded paw withdrawal latency to reflect the pain sensation. Injection of capsaicin alone (150 μM and 100 μl) caused initial acute pain, but after two hours when we started to measure heat induced paw lifting behavior, we observed no change in the paw withdrawal latency (FIG. 10C, solid circles in grey). After co-injection of MRS1477 and capsaicin (50 μM and 150 μM, respectively Total volume of 100 μl), we observed the paw withdrawal latency was increased in two hours, and such an analgesic effect diminished after three days (FIG. 10C, solid squares in green). With injection of our TAT-De3 and capsaicin (150 μM and 150 μM, respectively. Total volume of 100 μl), we found that our designed proteins excited analgesic effects to the similar magnitude like MRS1477 (FIG. 10C, solid squares in orange). Though the onset of such analgesic effects was later than that of MRS1477, after a single injection they lasted for about 16 days, which was much longer than that of MRS1477. Importantly, injection of PAMs (either MRS1477 or TAT-De3) did not cause hyperthemia in rats (FIG. 10D).

### VI. REFERENCES

1. Steglitz J, Buscemi J, & Ferguson M J (2012) The future of pain research, education, and treatment: a summary of the IOM report “Relieving pain in America: a blueprint for transforming prevention, care, education, and research”. Translational behavioral medicine 2(1):6-8.
2. Caterina M J, et al. (1997) The capsaicin receptor: a heat-activated ion channel in the pain pathway. Nature 389(6653):816-824.
3. Tominaga M, et al. (1998) The cloned capsaicin receptor integrates multiple pain-producing stimuli. Neuron 21(3):531-543.
4. Caterina M J, et al. (2000) Impaired nociception and pain sensation in mice lacking the capsaicin receptor. Science 288(5464):306-313.
5. Kort M E & Kyn P R (2012) TRPV1 antagonists: clinical setbacks and prospects for future development. Prog Med Chem 51:57-70.
6. Julius D (2013) TRP channels and pain. Annual review of cell and developmental biology 29:355-384.
7. Gavva N R (2009) Setbacks in the Clinical Development ofTRPV1 Antagonists. The Open Drug Discovery Journal 1:1-35.
8. Sapio M R, et al. (2018) Pain control through selective chemo-axotomy of centrally projecting TRPV1+ sensory neurons. The Journal of clinical investigation 128(4):1657-1670,
9. Tadarola M J & Mannes A J (2011) The vanilloid agonist resiniferatoxin for interventional-based pain control. Current topics in medicinal chemistry 11(17):2171-2179.
10. Roh E J, Keller J M, Olah Z, Iadarola M J, & Jacobson K A (2008) Structure-activity relationships of 1,4-dihydropyridines that act as enhancers of the vanilloid receptor 1 (TRPV1). Bioorganic & medicinal chemistry 16(20):9349-9358.
11. Lebovitz E E, et al. (2012) Positive allosteric modulation of TRPV1 as a novel analgesic mechanism. Molecular pain 8:70.
12. Kaszas K, et at (2012) Small molecule positive allosteric modulation of TRPV1 activation by vanilloids and acidic pH. The Journal of pharmacology and experimental therapeutics 340(1): 152-160.
13. Lishko P V, Procko E, Jin X, Phelps C B, & Gaudet R (2007) The ankyrin repeats of TRPV1 bind multiple ligands and modulate channel sensitivity. Neuron 54(6):905-918.
14. Cao E, Liao M, Cheng Y, & Julius D (2013) TRPV1 structures in distinct conformations reveal activation mechanisms. Nature 504(7478):113-118.
15. Liao M, Cao E, Julius D, & Cheng Y (2013) Structure of the TRPV1 ion channel determined by electron cryo-microscopy. Nature 504(7478):107-112.
16. Leaver-Fay A. et al. (2011) ROSETTA3: an object-oriented software suite for the simulation and design of macromolecules. Methods in enzymology 487:545-574.
17. Fleishman S J, et at. (2011) Computational design of proteins targeting the conserved stem region of influenza hemagglutinin. Science 332(6031):816-821.
18. Fleishman S J, et al. (2011) Hotspot-centric de novo design of protein binders. J Mol Biol 413(5):1047-1062.
19. Strauch E M, Fleishman S J, & Baker D (2014) Computational design of a pH-sensitive IgG binding protein. Proc Natl Acad Sci USA 111(2):675-680.
20. Phelps C B, Wang R R, Choo S S, & Gaudet R (2010) Differential regulation of TRPV1, TRPV3, and TRPV4 sensitivity through a conserved binding site on the ankyrin repeat domain. J Biol Chem 285(1):731-740.
21. Lau S Y, Procko E, & Gaudet R (2012) Distinct properties of Ca2+-calmodulin binding to N- and C-terminal regulatory regions of the TRPV1 channel. J Gen Physiol 140(5):541-555.
22. Gao Y, Cao E, Julius D, & Cheng Y (2016) TRPV1 structures in nanodiscs reveal mechanisms of ligand and lipid action. Nature 534(7607):347-351.
23. Schneidman-Duhovny D, Inbar Y, Nussinov R, & Wolfson H J (2005) PatchDock and SymmDock: servers for rigid and symmetric docking. Nucleic Acids Res 33(Web Server issue):W363-367.
24. Fleishman S J, et al. (2011) RosettaScripts: a scripting language interface to the Rosetta macromolecular modeling suite. PLoS One 6(6):e20161.
25. Huff J (2015) The Airyscan detector from ZEISS: confocal imaging with improved signal-to-noise ratio and super-resolution. Nature Methods 12(12):i-ii.
26. Takanishi C L, Bykova E A, Cheng W, & Zheng J (2006) GFP-based FRET analysis in live cells. Brain research 1091(1):132-139.
27. Zou L, Peng Q, Wang P. & Zhou B (2017) Progress in Research and Application of HIV-1 TAT-Derived Cell-Penetrating Peptide. The Journal of membrane biology 250(2):115-122.
28. Gaudet R (2008) A primer on ankyrin repeat function in TRP channels and beyond. Molecular bioSystems 4(5):372-379.
29. Stevens R M, et al. (2019) Randomized, Double-Blind, Placebo-Controlled Trial of Intraarticular Trans-Capsaicin for Pain Associated With Osteoarthritis of the Knee. Arthritis Rheumatol 71(9):1524-1533.
30. Cheng W, Yang F, Takanishi C L, & Zheng J (2007) Thermosensitive TRPV1 channel subunits coassemble into heteromeric channels with intermediate conductance and gating properties. J Gen Physiol 129(3):191-207.
31. Wang C, et al. (2007) RosettaDock in CAPRI rounds 6-12. Proteins 69(4):758-763.
32. Pettersen E F, et al. (2004) UCSF Chimera—a visualization system for exploratory research and analysis. Journal of computational chemistry 25(13):1605-1612.
33. Anonymous (2018) UCSF Chimera.
34. Qiu S, Hua Y L, Yang F, Chen Y Z, & Luo J H (2005) Subunit assembly of N-methyl-d-aspartate receptors analyzed by fluorescence resonance energy transfer. J Biol Chem 280(26):24923-24930.
35. Gavva N R, et al. (2005) AMG 9810 [(E)-3-(4-t-butylphenyl)-N-(2,3-dihydrobenzo[b][1,4]dioxin-6-yl)acrylamide], a novel vanilloid receptor 1 (TRPV1) antagonist with antihyperalgesic properties. The Journal of pharmacology and experimental therapeutics 313(1):474-484.

### VII. Exemplary Embodiments

Exemplary embodiments provided in accordance with the presently disclosed subject matter include, but are not limited to, the claims and the following embodiments:
1. A TRPV1 allosteric modulator comprising a peptide containing two hotspot amino acid residues,
wherein the hotspot amino acid residues are aromatic amino acid residues that interact with a TRPV1 ankyrin repeat domain (ARD), and
wherein alpha carbon atoms in the two hotspot amino acid residues are within 5-10 Å of each other upon folding of the peptide under physiological conditions.
2. The TRPV1 allosteric modulator of embodiment 1, comprising a polypeptide sequence having at least 70% identity to positions 64-94 of SEQ ID NO:9, provided that the amino acid residues at position 72 and position 76 are hotspot amino acid residues.
3. The TRPV1 allosteric modulator of embodiment 1 or embodiment 2, wherein the hotspot amino residues are phenylalanine residues.
4. The TRPV1 allosteric modulator of embodiment 2 or embodiment 3, comprising a polypeptide sequence having at least 95% identity to positions 64-94 of SEQ ID NO:9, provided that the amino acid residues at position 72 and position 76 are hotspot phenylalanine residues.
5. The TRPV1 allosteric modulator of any one of embodiments 2-4, comprising a polypeptide sequence having at least 70% identity to SEQ ID NO:9 provided that the amino acid residues at position 72 and position 76 are hotspot amino acid residues.
6. The TRPV1 allosteric modulator of embodiment 5, wherein the hotspot amino residues are phenylalanine residues.
7. The TRPV1 allosteric modulator of embodiment 6, comprising a polypeptide sequence having at least 90% identity to SEQ ID NO:9 provided that the amino acid residues at position 72 and position 76 are hotspot phenylalanine residues.
8. The TRPV1 allosteric modulator of any one of embodiments 1 and 5-7, wherein:
the peptide comprises a helix¹-loop-helix²-loop-helix³ architecture, helix¹, helix², and helix³ are folded in a three-helix bundle, and the hotspot amino acid residues are phenylalanine residues located in helix³.
9. The TRPV1 allosteric modulator of embodiment 1, comprising a polypeptide sequence having at least 70% identity to positions 35-89 of SEQ ID NO:8, provided that the amino acid residues at position 36 and position 83 are hotspot amino acid residues.
10. The TRPV1 allosteric modulator of embodiment 9, wherein the hotspot amino residues are phenylalanine residues.
11. The TRPV1 allosteric modulator of embodiment 9 or embodiment 10, comprising a polypeptide sequence having at least 95% identity to positions 35-89 of SEQ ID NO:8, provided that the amino acid residues at position 36 and position 83 are hotspot phenylalanine residues.
12. The TRPV1 allosteric modulator of any one of embodiments 9-11, comprising a polypeptide sequence having at least 70% identity to SEQ ID NO:8 provided that the amino acid residues at position 36 and position 83 are hotspot amino acid residues.
13. The TRPV1 allosteric modulator of embodiment 12, comprising a polypeptide sequence having at least 90% identity to SEQ ID NO:8 provided that the amino acid residues at position 36 and position 83 are hotspot phenylalanine residues.
14. The TRPV1 allosteric modulator of any one of embodiments 1, 12, and 13, wherein:
the peptide comprises a helix¹-loop-strand¹-helix²-turn-helix³-strand²-helix⁴ architecture,
strand¹ and strand² and folded in a parallel β sheet, and
the hotspot amino acid residues are phenylalanine residues located in helix² and helix⁴.
15. The TRPV1 allosteric modulator of any one of embodiments 1-14, further comprising a cell penetration peptide sequence.
16. The TRPV1 allosteric modulator of embodiment 15, wherein the cell penetration peptide sequence is an HIV-1 tat protein sequence.
17. A TRPV1 allosteric modulator prepared by process comprising:
i) assessing shape complementarity, binding energy, and solvent accessible surface area of a candidate peptide upon docking to a surface of a TRPV1 ankyrin repeat domain (ARD) in silico;
ii) assessing ab initio refolding of the candidate peptide:
iii) evolving the candidate peptide via in silico affinity maturation to define a mature allosteric modulator:
iv) expressing or synthesizing the mature allosteric modulator, thereby preparing the TRPV1 allosteric modulator.
18. The TRPV1 allosteric modulator of embodiment 17, wherein the process further comprises:
i-a) docking a surface of a peptide scaffold to a surface of the ARD to define a surface interface;
wherein the peptide scaffold comprises one or more ARD-binding hotspot amino acid residues and a plurality of non-hotspot amino acid residues;
i-b) fixing the hotspot amino acid residues to a target location on the surface of the TRPV1 ARD;
i-c) substituting at least one non-hotspot residue with another amino acid residue to define the candidate peptide.
19. The TRPV1 allosteric modulator of embodiment 18, wherein the hotspot amino acid residues are phenylalanine residues.
20. The TRPV1 allosteric modulator of embodiment 18 or embodiment 19, wherein the target location on the surface of the TRPV1 ARD is located on the concave surface formed by the 2nd and 3rd fingers of the ARD.
21. The TRPV1 allosteric modulator of any one of embodiments 18-20, wherein the peptide scaffold is characterized by one or more properties selected from the group consisting of:
an X-ray crystal structure determined at a resolution of at least 2.5 Å;
an absence of DNA, RNA, or disulfide bonds in the X-ray crystal structure;
an absence of ligands or cofactors in the X-ray crystal structure;
no more than 100 amino acid residues in the primary amino acid sequence;
no more than one polypeptide chain;
monomeric stoichiometry; and
previous expression in E. coli reported.
22. The TRPV1 allosteric modulator of any one of embodiments 18-21, wherein:
the peptide scaffold comprises a helix¹-loop-strand¹-helix²-turn-helix³-strand²-helix⁴ architecture,
strand¹ and strand² and folded in a parallel β sheet, and
the hotspot amino acid residues are phenylalanine residues located in helix² and helix⁴.
23. The TRPV1 allosteric modulator of any one of embodiments 18-21, wherein:
the peptide scaffold comprises a helix¹-loop-helix²-loop-helix³ architecture,
helix¹, helix², and helix³ are folded in a three-helix bundle, and
the hotspot amino acid residues are phenylalanine residues located in helix³.
24. A TRPV1 allosteric modulator comprising a polypeptide sequence having at least 70% identity to positions 64-94 of SEQ ID NO:9, provided that the amino acid residues at position 72 and position 76 are aromatic amino acid residues.
25. The TRPV1 allosteric modulator of embodiment 24, wherein the aromatic amino residues are phenylalanine residues.
26. The TRPV1 allosteric modulator of embodiment 24 or embodiment 25, comprising a polypeptide sequence having at least 95% identity to positions 64-94 of SEQ ID NO:9, provided that the amino acid residues at position 72 and position 76 are phenylalanine residues.
27. The TRPV1 allosteric modulator of any one of embodiments 24-26, comprising a polypeptide sequence having at least 70% identity to SEQ ID NO:9 provided that the amino acid residues at position 72 and position 76 are aromatic amino acid residues.
28. The TRPV1 allosteric modulator of embodiment 27, wherein the aromatic amino residues are phenylalanine residues.
29. The TRPV1 allosteric modulator of embodiment 28, comprising a polypeptide sequence having at least 90% identity to SEQ ID NO:9 provided that the amino acid residues at position 72 and position 76 are phenylalanine residues.
30. A TRPV1 allosteric modulator comprising a polypeptide sequence having at least 70% identity to positions 35-89 of SEQ ID NO:8, provided that the amino acid residues at position 36 and position 83 are aromatic amino acid residues.
31. The TRPV1 allosteric modulator of embodiment 30, wherein the aromatic amino residues are phenylalanine residues.
32. The TRPV1 allosteric modulator of embodiment 30 or embodiment 31, comprising a polypeptide sequence having at least 95% identity to positions 35-89 of SEQ ID NO:8, provided that the amino acid residues at position 36 and position 83 are hotspot phenylalanine residues.
33. The TRPV1 allosteric modulator of any one of embodiments 30-32, comprising a polypeptide sequence having at least 70% identity to SEQ ID NO:8 provided that the amino acid residues at position 36 and position 83 are aromatic amino acid residues.
34. The TRPV1 allosteric modulator of embodiment 33, comprising a polypeptide sequence having at least 90% identity to SEQ ID NO:8 provided that the amino acid residues at position 36 and position 83 are phenylalanine residues.
35. A nucleic acid comprising a polynucleotide sequence encoding a TRPV1 allosteric modulator according to any one of embodiments 1-34.
36. An expression cassette comprising the nucleic acid of embodiment 35.
37. A recombinant cell comprising the nucleic acid of embodiment 35 or the expression cassette of embodiment 36.
38. A pharmaceutical composition comprising a TRPV1 allosteric modulator according to any one of embodiments 1-34 or the nucleic acid of embodiment 35 and a pharmaceutically acceptable excipient.
39. A method for treating a condition associated with TRPV1 activity, the method comprising administering an effective amount of a TRPV1 allosteric modulator according to any one of embodiments 1-34 or an effective amount of a pharmaceutical composition of embodiment 38 to a subject in need thereof.
40. The method of embodiment 39, wherein the condition is selected from the group consisting of pain, pruritus, and cancer.
41. The method of embodiment 39, wherein the pain comprises chronic pain or acute pain.
42. The method of embodiment 40 or embodiment 41, wherein the pain comprises neuropathic pain, inflammatory pain, cancer pain, or a combination thereof.
43. The method of any one of embodiments 39-42, further comprising administering; a non-steroidal anti-inflammatory agent, an opioid analgesic, acetaminophen, or a combination thereof.
44. The method of embodiment 40, wherein the pruritus comprises histamine-induced itching, lymphoma-induced itching, allergic itching, infection-induced itching, liver- or kidney-induced itching, diabetes-induced itching, skin disorder-induced itching, opioid-induced itching, or a combination thereof.
45. The method of any one of embodiments, 39, 40, and 44, further comprising administering an antihistamine, a steroid, an anesthetic, or a combination thereof to the subject.
46. The method of embodiment 40, wherein the cancer is breast cancer, squamous cell carcinoma, hepatocellular carcinoma, or carcinoma of the bladder.
47. The method of any one of embodiments 39, 40, and 46, further comprising administering an anti-cancer agent to the subject.

Although the foregoing has been described in some detail by way of illustration and example for purposes of clarity and understanding, one of skill in the art will appreciate that certain changes and modifications can be practiced within the scope of the appended claims. In addition, each reference provided herein is incorporated by reference in its entirety to the same extent as if each reference was individually incorporated by reference.

INFORMAL SEQUENCE LISTING

SEQ ID NO: 1
SGYTEDEKLNIAKRHLLPKQIERNALKKGELTVDDSAIIGIIRYYTREAGVRGLEREISKLCRK
AVKQLLLDKSLKHIEINGDNLHDYLGVQRF
SEQ ID NO: 2
SLLISYESDFKTTLEQAKASLAEAPSQPLSQRNTTLKHVEQQQDELFDLLDQMDVEVNNSIGD
ASERATYKAKLREWKKTIQSDIKRPLQSLVDSGD
SEQ ID NO: 3
MVYQVKDKADLDGQLTKASGKLVVLDFFATWCGPCKMISPKLVELSTQFADNVVVLKVDV
DECEDIAMEYNISSMPTFVFLKNGVKVEEFAGANAKRLEDVIKANI
SEQ ID NO: 4
GHMIKGEQKRYSEMTKEELQQEIAMLTEKARKAEQMGMVNEYAVYERKIAMAKAYMLNP
ADFHPGEIYEIEGAPGEYFKVRYLKGVFAWGWRLKGNGEEEALPISLLRKPNLPQS
SEQ ID NO: 5
MIPSITAYSKNGLKIEFTFERSNTNPSVTVITIQASNSTELDMTDFVFQAAVPKTFQLQELSPSS
SVVPAFNTGTITQVIKVLNPQKQQLRMRIKLTYNHKGSAMQDLAEVNNFPPQSWQ
SEQ ID NO: 6
MDETGKELVLVLYDYQEKSPRELTVKKGDILTLLNSTNKDWWKIEVNGRQGFVPAAYLKKLD
SEQ ID NO: 7
MGKLPESPKRAEEIWQQSVIGDYLARFKNDRVKALKAMEMTWNNMEKKEKLMWIKKAAE
DQKRYERELSEMRAPPAATNSSKKLE
SEQ ID NO: 8 (De1)
NGYSSEDKLRIAKRHLLPKQIERNALKKGELFVSEFAILGIILFYTDEAGVRGLEREISKLCRKA
VKQLLLDKSTTSSHASGFHLHDYLGVMR
SEQ ID NO: 9 (De3)
SLLDSYKLDFWKTSTNAENSLHEAPSQPLSQRNTTLKHVEQQQDELFDLLDQMDVEVNNSIG
RDADRRWYLFELWFWKISIEEEIKRPLQSLVDSG
SEQ ID NO: 10 (15237)
SLLDSYKLDFWKTSTNAENSLHEAPSQPLSQRNTTLKHVEQQQDELFDLLDQMDVE
VNNSIGRDADRRWYLFELWFWKISIEEEIKRPLQSLVDSG
SEQ ID NO: 11 (15237_2nd_0086)
SLLISYRDDFFTTFWQATQSLKEAPSQPLSQRNTTLKHVEQQQDELFDLLDQMDVEV
NNSIGSLASKFAYKFLLDEWKDDIQRDIKRPLQSLVDSG
SEQ ID NO: 12 (5132)
MVYQAKDFADLFGQAANASGKLIVTVLFATWCGPCKMISPKLVELSTQFADNVVVI
KVKALKFEEFARDNNLSSMPVLVFWKNGDEVESFAGANAKRLEDVIKAN
SEQ ID NO: 13 (11577)
EQKRYSEMTKEELQQEIAMLTEKARKAEQMGMVNEYAVYERKIAMAKAYMLNPA
DFHPGEIYEIEGAPGEYFKVRYLKGVFAWGWRLKGNGEEEALPISLLRK
SEQ ID NO: 14 (2824)
NGYSSEDKLRIAKRHLLPKQIERNALKKGELFVSEFAILGIILFYTDEAGVRGLEREISK
LCRKAVKQLLLDKSTTSSHASGFHLHDYLGVMR
SEQ ID NO: 15 (926)
MIPSITAYSKNGLKIEFTFERSNTNPSVTVITIQASNSTELDMTDFVFQAAVPKTFQLQ
ELSPSSSVVPAFNTGTITQVIKVLNPQKQQLRMRIKLTYNHKGSAMQDLAEVNNFPP
QSW
SEQ ID NO: 16 (15080)
KELVLVLEEFKSHAPRSLKVKKGDILTILNDDDFSNAKIEVNGRQGFVPLFFAHRI
SEQ ID NO: 17 (15080_cst)
KLLVLVYDYYSATAPRELTVKKGDILTLLNADLMFYLKIEVNGRQGFVPAFFALWM
SEQ ID NO: 18 (8320_2nd_0141)
LPESPKRAEEIWQQSVIGDYLARFKNDRDRAYASMEFHWRFMSEDLKLMWIKKAAE
DQKRYEREL
SEQ ID NO: 19
RKKRRQRRR
SEQ ID NO: 20
YGRKKRRQRRRA
SEQ ID NO: 21
GRKKRRQRRRPPQ
SEQ ID NO: 22
RQIKIWFQNRRMKWKK
SEQ ID NO: 23
VKRGLKLRHVRPRVTRMDV
SEQ ID NO: 24
GALFLGFLGAAGSTMGAWSQPKKKRKV
SEQ ID NO: 25
KETWWETWWTEWSQPKKKRKV
SEQ ID NO: 26
LLIILRRRIRKQAHAHSK
SEQ ID NO: 27
MVRRFLVTLRIRRACGPPRVRV
SEQ ID NO: 28
MVKSKIGSWILVLFVAMWSDVGLCKKRP
SEQ ID NO: 29
KLALKLALKALKAALKLA
SEQ ID NO: 30
GWTLNSAGYLLGKINLKALAALAKKIL
SEQ ID NO: 31
LSTAADMQGVVTDGMASGLDKDYLKPDD
SEQ ID NO: 32
DPKGDPKGVTVTVTVTVTGKGDPKPD
SEQ ID NO: 33
RRIRPRPPRLPRPRPRPLPFPRPG
SEQ ID NO: 34
CSIPPEVKFNKPFVYLI
SEQ ID NO: 35
PFVYLI
SEQ ID NO: 36
SDLWEMMMVSLACQY
SEQ ID NO: 37
NGYWSEDKERIAKRHLLPKQIERNALKKGELKVWEFAIFGIILFYTWEAGVRGLEREI
SKLCRKAVKQLLLDKSTTRSWASGFHLHDYLGVMR
SEQ ID NO: 38
LPESPRFAQKIWQQSVIGDYLARFKNDRVKALKAMEDSSNNSSMHRFAEFFAKAFED
LHRYLEEL
SEQ ID NO: 39
MSVSAGEKPPRLYDRRSIFDAVAQSNCQELESLLPFLQRSKKRLTDSEFKDPETGKTC
LLKAMLNLHNGQNDTIALLLDVARKTDSLKQFVNASYTDSYYKGQTALHIAIERRN
MTLVTLLVENGADVQAAANGDFFKKTKGRPGFYFGELPLSLAACTNQLAIVKFLLQ
NSWQPADISARDSVGNTVLHALVEVADNTVDNTKFVTSMYNEILILGAKLHPTLKLE
EITNRKGLTPLALAASSGKIGVLAYILQREIHEPECRHAAAHHHHHH
SEQ ID NO: 40
LYDRRSIFDAVAQSNCQELESLLPFLQRSKKRLTDSEFKDPETGKTCLLKAMLNLHN
GQNDTIALLLDVARKTDSLKQFVNASYTDSYYKGQTALHIAIERRNMTLVTLLVENG
ADVQAAANGDFFKKTKGRPGFYFGELPLSLAACTNQLAIVKFLLQNSWQPADISARD
SVGNTVLHALVEVADNTVDNTKFVTSMYNEILILGAKLHPTLKLEEITNRKGLTPLA
LAASSGKIGVLAYILQREIH

What is claimed is:

1. A vanilloid receptor 1 (TRPV1) allosteric modulator comprising a peptide containing two hotspot amino acid residues,
wherein the hotspot amino acid residues are aromatic amino acid residues that interact with a TRPV1 ankyrin repeat domain (ARD), and
wherein alpha carbon atoms in the two hotspot amino acid residues are within 5-10 Å of each other upon folding of the peptide under physiological conditions.

2. The TRPV1 allosteric modulator of claim 1, wherein the hotspot amino residues are phenylalanine residues.

3. The TRPV1 allosteric modulator of claim 1, comprising a polypeptide sequence having at least 70% identity to positions 64-94 of SEQ ID NO:9, provided that the amino acid residues at position 72 and position 76 are hotspot amino acid residues.

4. The TRPV1 allosteric modulator of claim 3, comprising a polypeptide sequence having at least 95% identity to positions 64-94 of SEQ ID NO:9, provided that the amino acid residues at position 72 and position 76 are hotspot phenylalanine residues.

5. The TRPV1 allosteric modulator of claim 3, comprising a polypeptide sequence having at least 70% identity to SEQ ID NO:9 provided that the amino acid residues at position 72 and position 76 are hotspot amino acid residues.

6. The TRPV1 allosteric modulator of claim 5, wherein the hotspot amino residues are phenylalanine residues.

7. The TRPV1 allosteric modulator of claim 6, comprising a polypeptide sequence having at least 90% identity to SEQ ID NO:9 provided that the amino acid residues at position 72 and position 76 are hotspot phenylalanine residues.

8. The TRPV1 allosteric modulator of claim 7, wherein:
the peptide comprises a helix¹-loop-helix²-loop-helix³ architecture,
helix¹, helix², and helix³ are folded in a three-helix bundle, and
the hotspot amino acid residues are phenylalanine residues located in helix³.

9. The TRPV1 allosteric modulator of claim 1, comprising a polypeptide sequence having at least 70% identity to positions 35-89 of SEQ ID NO:8, provided that the amino acid residues at position 36 and position 83 are hotspot amino acid residues.

10. The TRPV1 allosteric modulator of claim 9, wherein the hotspot amino residues are phenylalanine residues.

11. The TRPV1 allosteric modulator of claim 9, comprising a polypeptide sequence having at least 95% identity to positions 35-89 of SEQ ID NO:8 provided that the amino acid residues at position 36 and position 83 are hotspot phenylalanine residues.

12. The TRPV1 allosteric modulator of claim 11, comprising a polypeptide sequence having at least 70% identity to SEQ ID NO:8 provided that the amino acid residues at position 36 and position 83 are hotspot amino acid residues.

13. The TRPV1 allosteric modulator of claim 12, comprising a polypeptide sequence having at least 90% identity to SEQ ID NO:8 provided that the amino acid residues at position 36 and position 83 are hotspot phenylalanine residues.

14. The TRPV1 allosteric modulator of claim 13, wherein:
the peptide comprises a helix¹-loop-strand¹-helix²-turn-helix³-strand²-helix⁴ architecture,
strand¹ and strand² and folded in a parallel β sheet, and
the hotspot amino acid residues are phenylalanine residues located in helix² and helix⁴.

15. The TRPV1 allosteric modulator of claim 1, further comprising a cell penetration peptide sequence.

16. The TRPV1 allosteric modulator of claim 15, wherein the cell penetration peptide sequence is an HIV-1 tat protein sequence.

17. A nucleic acid comprising a polynucleotide sequence encoding a TRPV1 allosteric modulator according to any one of claims 1-16.

18. An expression cassette comprising the nucleic acid of claim 17.

19. A recombinant cell comprising the nucleic acid of claim 17 or the expression cassette of claim 18.

20. A pharmaceutical composition comprising a TRPV1 allosteric modulator according to any one of claims 1-16 or the nucleic acid of claim 17 and a pharmaceutically acceptable excipient.

21. A method for treating a condition associated with TRPV1 activity, the method comprising administering an effective amount of a TRPV1 allosteric modulator according to any one of claims 1-16 to a subject in need thereof.

22. The method of claim 21, wherein the condition is selected from the group consisting of pain, pruritus, and cancer.

23. The method of claim 22, wherein the pain comprises chronic pain or acute pain.

24. The method of claim 22, wherein the pain comprises neuropathic pain, inflammatory pain, cancer pain, or a combination thereof.

25. The method of claim 24, further comprising administering a non-steroidal anti-inflammatory agent, an opioid analgesic, acetaminophen, or a combination thereof to the subject.

26. The method of claim 22, wherein the pruritus comprises histamine-induced itching, lymphoma-induced itching, allergic itching, infection-induced itching, liver- or kidney-induced itching, diabetes-induced itching, skin disorder-induced itching, opioid-induced itching, or a combination thereof.

27. The method of claim 22, further comprising administering an antihistamine, a steroid, an anesthetic, or a combination thereof to the subject.

28. The method of claim 22, wherein the cancer is breast cancer, squamous cell carcinoma, hepatocellular carcinoma, or carcinoma of the bladder.

29. The method of claim 22, further comprising administering an anti-cancer agent to the subject.
